(19) United States
(12) Patent Application Publication
(10) Pub. No.: US 2025/0281530 A1
(43) Pub. Date: Sep. 11, 2025

(54) METHODS OF PREPARING COHN POOL CONCENTRATE FROM BLOOD PLASMA THROUGH ULTRAFILTRATION

(71) Applicant: Takeda Pharmaceutical Company Limited, Osaka (JP)

(72) Inventors: Ivano Martelli, Rome (IT); Oliver Mieselberger, Vienna (AT); Francesca Micheli, Rieti (IT); Alexander Zaydenberg, Chatsworth, CA (US); Jarrod Hanson, Covina, CA (US)

(21) Appl. No.: 18/862,502

(22) PCT Filed: May 1, 2023
(86) PCT No.: PCT/US23/66457

§ 371 Date: Nov. 1, 2024

Related U.S. Application Data
(60) Provisional application No. 63/337,439, filed on May 2, 2022.
(60) Provisional application No. 63/498,470, filed on Apr. 26, 2023.

(51) Int. Cl. A61K 35/16

(52) U.S. Cl. CPC A61K 35/16

(57) ABSTRACT

The present invention provides a method of fractionating human plasma, in some embodiments, using the Cohn fractionation procedure. The improvement comprises the use of physiologically active concentrated plasma as the starting material for the fractionation procedure.

### FIELD OF THE INVENTION

The present invention resides in the field of plasma fractionation to separate therapeutically active plasma proteins from plasma.

### BACKGROUND OF THE INVENTION

The past decade has seen a steady increase in the clinical utilization of plasma protein-based therapeutics. For example, as awareness of primary immunodeficiency disease (PID) has increased, the effective clinical use of intravenous immunoglobulin (IVIG) has increased across the patient population affected by PID. IVIG is increasingly being utilized for off-label indications as well for conditions such as chronic inflammatory demyelinating polyneuropathy (CIPD).

In 2019, the blood plasma product market was forecast to grow at a CAGR of 6.8% to reach $28.5 B in 2023 from $20.5 B in 2018. The global annual fractionation capacity was about 70.7 million liters in 2016.

The process of plasma fractionation is infrastructure/facility intensive and highly regulated. To meet the increasing demand for plasma-derived therapeutics, either existing infrastructure must be capable of meeting this demand, or the infrastructure must be modified or increased with the latter two of these options requiring significant capital outlay, a potential interruption to the production line and potential regulatory review and certification of the modified or new facilities. Thus, an option for enhancing the efficiency of existing infrastructure without its substantial modification is highly attractive.

A method of reducing the volume of liquid plasma input into the fractionation process could achieve the dual benefits of maximizing the productivity of existing fractionation infrastructure as well as reduce the level of needed resources during plasma fractionation. A method apparently unexplored until the present invention is one in which a reduced volume of liquid is processed to produce the same amount of plasma-derived protein product as would be produced from a higher volume of liquid, e.g., Fresh-Frozen Plasma (FFP).

Accordingly, until the invention described herein, it has not been apparent that the proteins in the various fractions (e.g., cold ethanol fractions) could be recovered by fractionating a concentrated plasma Cohn Pool in amounts sufficiently meaningful to make the expense of concentrating and fractionating the concentrated physiologically active plasma worthwhile. Additionally, it was not known whether the concentrated plasma Cohn Pool would act similarly to fresh frozen plasma in Cohn Fractionation (or a known modification thereof). The inventors have discovered that a fractionation route originating with a concentrated plasma Cohn Pool is indeed feasible. The concentrated Cohn Pool is a component of an economically viable fractionation process, e.g., Cohn Fractionation or Kistler-Nitschman Fractionation, or other method (e.g., Gerlough, Hink, and Mulford methods) commencing with a concentrated plasma Cohn Pool. See, e.g., Kistler et al., Vox. Sang. (1962); 7(4), pp. 414-424; Graham, et al. Subcellular Fractionation, a Practical Approach. Oxford University Press. 1997.

### BRIEF SUMMARY OF THE INVENTION

Given the increasingly broad use of therapeutic plasma-derived blood protein compositions, such as immune globulin compositions, albumin, protease inhibitors, blood coagulation factors, coagulation factor inhibitors, and proteins of the complement system, ensuring adequate, economical, environmentally friendly, and sustainable access to efficacious and safe plasma-derived blood protein compositions is of paramount importance.

It has now been discovered that modifying a standard plasma protein fractionation process by inputting into the process a concentrated plasma Cohn Pool leads to process efficiencies consequent to reducing the volume of liquid to be fractionated and, in some embodiments, surprisingly, essentially proportional to the increase in concentration of the plasma input introduced into the process. The improved method results in increased utilization of processing equipment and an increase in the throughput, which, in some embodiments, is roughly proportional to the concentration factor and can, therefore, be significant. In various embodiments, the invention provides an improved plasma fractionation method having one or more of these improved properties. Also provided are plasma protein products prepared using the improved procedure.

With the current invention, it has been discovered that a concentrated plasma Cohn Pool is an efficacious starting material for preparing protein therapeutic agents by fractionating the concentrated Cohn Pool. In various embodiments, the proteins typically found in the various Cohn fractions downstream from the concentrated plasma Cohn Pool are found in these fractions in yields and purity comparable to those in which they found in corresponding fractions in a process starting with a (non-concentrated) plasma feedstock, e.g., cryo poor plasma, plasma following one or more absorptive steps, recovered plasma, plasma from plasmapheresis, frozen plasma, thawed plasma and the like.

An exemplary method of the invention includes: (a) submitting a concentrated plasma Cohn Pool to one or more plasma fractionation processes (e.g., cold ethanol fractionation). In an exemplary embodiment, the invention provides, prior to (a), (b) preparing a concentrated plasma Cohn Pool.

Thus, in various embodiments, the invention provides an improved process for fractionating plasma. The improvement comprises initiating the plasma fractionation process with a concentrated plasma Cohn Pool. In various embodiments, the improvement further comprises concentrating a plasma input prior to submitting the concentrated input to a first alcohol fractionation step. An exemplary plasma input is concentrated cryo poor plasma.

In various embodiments, the reduction in plasma input volume and concomitant increase in plasma protein concentration(s) results in a plasma fractionation method that is, to a surprising degree characterized by the reduction in volume/increase in plasma protein concentration(s).

In various embodiments, the invention provides an improved plasma fractionation process proceeding to completion of a selected step within a determined amount of time. The improvement to the plasma fractionation process comprises completing the selected final step in a time reduced relative to a plasma fractionation process otherwise identical with the exception that the plasma input into the first alcohol fractionation step in the method of the invention is concentrated relative to the plasma input into the otherwise identical process. An exemplary plasma input is concentrated cryo poor plasma, thus, concentrated cryo poor plasma is the concentrated Cohn Pool in this embodiment.

For example, a fractionation process initiated with a Cohn Pool concentrated by about 10%, about 20% or about 30% relative to a standard plasma fractionation input results in the use of about 10%, about 20% or about 30% less reagents than a comparable process beginning with an unconcentrated input. Similarly, a fractionation process initiated with a Cohn Pool concentrated by about 10%, about 20% or about 30% relative to a standard plasma fractionation input results in the expenditure of about 10%, about 20% or about 30% less time from start to completion than a comparable process beginning with an unconcentrated input. This result was not expected as the mass of the plasma proteins in the concentrated input is essentially unchanged between the concentrated and unconcentrated inputs.

In an exemplary embodiment, the invention provides an improved method of preparing a protein fraction from plasma, wherein the fraction is enriched in a plasma protein product selected from a coagulation factor (e.g., factor V, VII, VIII, IX, X, XI, XII, and XIII), the prothrombin complex, Von Willebrand factor, factor VIII/Von Willebrand factor, fibrin, fibrinogen, thrombin, polyvalent and hyperimmune (such as anti-RhO, anti-hepatitis B, anti-rabies, or anti-tetanus) immunoglobulins (IgGs), protease inhibitors (such as alpha 1-antitrypsin and C1-inhibitor), anticoagulants (such as antithrombin), C1-esterase inhibitor, Protein C, and albumin, and a combination thereof. The improvement comprises, concentrating a plasma input, preparing a concentrated Cohn Pool, prior to introducing the concentrated Cohn Pool to a first alcohol fractionation step of a plasma fractionation method. An exemplary plasma input is concentrated cryo poor plasma.

The instant improved process is applicable to any plasma fractionation process, e.g., Cohn, Gerlough, Hink, Mulford, Kistler Nitschmann, heat ethanol fractionation, Hao, etc.

The present invention also provides a plasma processing system, preferably a cGMP compliant system, which is used, inter alia, to fractionate plasma from a concentrated Cohn pool input, e.g., concentrated cryo poor plasma.

### BRIEF DESCRIPTION OF THE DRAWINGS

FIG. 1 is a generalized flow diagram of an exemplary Cohn fractionation procedure.

FIG. 2 is an exemplary flow diagram for concentrating starting plasma to form the concentrated Cohn pool input into the fractionation process.

FIG. 3 illustrates an exemplary improved fractionation process of the invention with the filtration step identified and located with a red dot.

### DETAILED DESCRIPTION OF THE INVENTION

### I. Introduction

Making up about 55% of the total volume of whole blood, blood plasma is a whole blood component in which blood cells and other constituents of whole blood are suspended. Blood plasma further contains a mixture of over 700 proteins and additional substances that perform functions necessary for bodily health, including clotting, protein storage, and electrolytic balance, amongst others. When extracted from whole blood, blood plasma may be employed to replace bodily fluids, antibodies and clotting factors. Accordingly, blood plasma is extensively used in medical treatments.

As set forth hereinabove and in the following sections, the present invention, by starting fractionation with a concentrated plasma Cohn Pool, imparts numerous efficiencies and other advantages to the fractionation process.

Reference will now be made in detail to implementation of exemplary embodiments of the present disclosure as illustrated in the accompanying drawings. The same reference indicators will be used throughout the drawings and the following detailed description to refer to the same or like parts. Those of ordinary skill in the art will understand that the following detailed description is illustrative only and is not intended to be in any way limiting. Other embodiments of the present disclosure will readily suggest themselves to such skilled persons having benefit of this disclosure.

In the interest of clarity, not all of the routine features of the implementations described herein are shown and described. It will be appreciated that, in the development of any such actual implementation, numerous implementation-specific decisions are made in order to achieve the plasma product producer's specific goals, such as compliance with application-and business-related constraints, and that these specific goals will vary from one implementation to another and from one plasma product producer to another. Moreover, it will be appreciated that such a development effort might be complex and time-consuming, but would nevertheless be a routine undertaking of engineering for those of ordinary skill in the art having the benefit of this disclosure.

Many modifications and variations of the exemplary embodiments set forth in this disclosure can be made without departing from the spirit and scope of the exemplary embodiments, as will be apparent to those skilled in the art. The specific exemplary embodiments described herein are offered by way of example only, and the disclosure is to be limited only by the terms of the appended claims, along with the full scope of equivalents to which such claims are entitled.

### II. Abbreviations and Definitions

Unless defined otherwise, all technical and scientific terms used herein generally have the same meaning as commonly understood by one of ordinary skill in the art to which this invention belongs. Generally, the nomenclature used herein and the laboratory procedures in organic chemistry, pharmaceutical formulation, and medical imaging are those well-known and commonly employed in the art.

### b. Definitions

The articles “a” and “an” are used herein to refer to one or to more than one (i.e., to at least one) of the grammatical object of the article. By way of example, “a protein” means one protein or more than one protein.

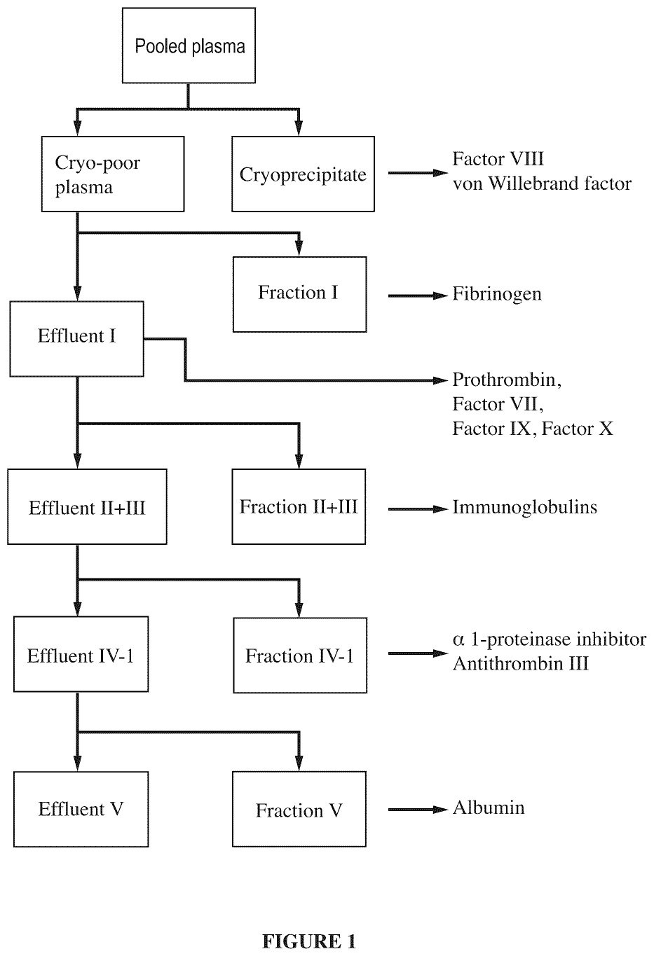

The “Cohn Process”, and “Cohn Fractionation” are used interchangeably herein and as generally understood, refer to a method of separating human plasma through a series of steps, including ethanol precipitation at differing concentrations, changes in pH, changes in temperature, changes in ionic strength, which lead to fractions enriched in certain plasma proteins. See, for example U.S. Pat. No, 2,390,074. FIG. 1 provides an exemplary flow diagram for the Cohn Process. As used herein, the terms “Cohn Process” and “Cohn Fractionation” also refers to the many variations and improvements on this pioneering process, e.g., Kistler-Nitschmann Process (Kistler et al. (1952), Vox Sang, 7, 414-424). Other processes of use in the methods of the invention include the method of isolating IgG set forth in U.S. Pat. No. 8,940,877

“Plasma” is the fluid that remains after blood has been centrifuged (for example) to remove cellular materials such as red blood cells, white blood cells and platelets. Plasma is generally yellow-colored and clear to opaque. Blood that is donated and processed to separate the plasma from the other certain blood components, and not frozen is referred to as “never-frozen” plasma. Plasma that is frozen within 8 hours to temperatures, described herein, is referred to herein as “fresh frozen plasma” (“FFP”). It contains the dissolved constituents of the blood such as proteins (6-8%; e.g., serum albumins, globulins, fibrinogen, etc.), glucose, clotting factors (clotting proteins), electrolytes (Na⁺, Ca²⁺, Mg²⁺, HCO₃⁻, Cl⁻, etc.), hormones, etc. Whole blood (WB) plasma is plasma isolated from whole blood with no added agents except anticoagulant(s). Citrate phosphate dextrose (CPD) plasma, as the name indicates, contains citrate, sodium phosphate and a sugar, usually dextrose, which are added as anticoagulants.

“Recovered plasma” refers to plasma separated no later than 5 days after the expiration date of the Whole Blood and is stored at 1 to 6° C. The profile of plasma proteins in Liquid Plasma is poorly characterized. Levels and activation state of coagulation proteins in Liquid Plasma are dependent upon and change with time in contact with cells, as well as the conditions and duration of storage. This component serves as a source of plasma proteins. Levels and activation state of coagulation proteins are variable and change over time.

“Thawed plasma” refers to plasma derived from Source, FFP or FP24, prepared using aseptic techniques (closed system), thawed at from about 10 to about 37° C., and maintained at from about 1 to about 6° C. for up to about 4 days after the initial 24-hour post-thaw period has elapsed. Thawed plasma contains stable coagulation factors such as Factor II and fibrinogen in concentrations similar to those of FFP, but variably reduced amounts of other factors. An exemplary thawed plasma is a component of the first fractionation step where the plasma (source, Recovered, FF etc) is removed from plastic containers and thawed in a jacketed vessel (with an exchange fluid at the temperature up 37° C.)

“Fresh frozen plasma” (“FFP”) refers to plasma prepared from a whole blood or apheresis collection and frozen at about −18° C. or colder within the time frame as specified in the directions for use for the relevant blood collection, processing, and storage system (e.g., frozen within eight hours of draw). On average, units contain 200 to 250 mL, but apheresis derived units may contain as much as 400 to 600 mL. FFP contains plasma proteins including all coagulation factors. FFP contains high levels of the labile coagulation Factors V and VIII.

As used herein, a “Factor” followed by a Roman Numeral refers to a series of plasma proteins which are related through a complex cascade of enzyme-catalyzed reactions involving the sequential cleavage of large protein molecules to produce peptides, each of which converts an inactive zymogen precursor into an active enzyme leading to the formation of a fibrin clot. They include: Factor I (fibrinogen), Factor II (prothrombin), Factor III (tissue thromboplastin), Factor IV (calcium), Factor V (proaccelerin), Factor VI (no longer considered active in hemostasis), Factor VII (proconvertin), Factor VIII (antihemophilic factor), Factor IX (plasma thromboplastin component; Christmas factor), Factor X (Stuart factor), Factor XI (plasma thromboplastin antecedent), Factor XII (hageman factor), and Factor XIII (fibrin stabilizing factor).

A “plasma protein” includes, for example, coagulation factors (such as factor V, VII, VIII, IX, X, XI, XII, and XIII), the prothrombin complex, Von Willebrand factor, factor VIII/Von Willebrand factor, fibrin, fibrinogen, thrombin, polyvalent and hyperimmune (such as anti-RhO, anti-hepatitis B, anti-rabies, or anti-tetanus) immunoglobulins (IgGs), protease inhibitors (such as alpha 1-antitrypsin and C1-inhibitor), anticoagulants (such as antithrombin), C1-esterase inhibitor, Protein C, and albumin, and a combination thereof.

As used herein, the term concentrated plasma “Cohn Pool” refers to a plasma pool, which, in the method of the invention, is concentrated and subsequently undergoes a plasma fractionation process (e.g., Cryo poor, Coagulation Factors poor, Inhibitors poor plasma). An exemplary concentrated plasma Cohn Pool is a physiologically active plasma concentrated by from about 10% to about 30% from its original volume (e.g., volume upon collection from a donor or donor pool, receipt by a fractionation facility, etc.), and which includes proteins that have not been damaged to such an extent to lose substantially all of their physiological activity.

In an exemplary embodiment, the concentration process results in essentially no diminution in the activity of a selected plasma protein subsequently isolated (or enriched) by fractionation of the concentrated Cohn Pool, e.g., IgG, A1PI, a Factor, etc. In various embodiments, the activity of a selected plasma protein fractionated from the physiologically active concentrated Cohn Pool is not less than about 99%, not less than abut 90%, not less than about 85% or not less than about 80% of the activity of the selected plasma protein in plasma feedstock before its concentration. In various embodiments, the selected plasma protein is polyvalent or hyperimmune IgG.

A “disease” is a state of health of an animal wherein the animal cannot maintain homeostasis, and wherein if the disease is not ameliorated then the animal's health continues to deteriorate. In various embodiments, one or more proteins from the fractionated physiologically active concentrated Cohn Pool are used to treat one or more disease.

### III. Embodiments

### A. Compositions and Devices

Embodiments of the present disclosure are directed to methods of concentrating a physiologically active plasma feedstock to form a concentrated Cohn Pool and subsequently fractionating the resulting physiologically active concentrated Cohn Pool using an art-recognized fractionation process. An exemplary fractionation process includes as its first step an alcohol fractionation step. Also provided are plasma protein preparations prepared by a fractionation process commencing with the concentrated Cohn Pool.

In an exemplary embodiment, the invention provides a physiologically active concentrated plasma. The biological and physiological activity of the concentrated plasma is essentially non-degraded when compared to the starting plasma from which the concentrated plasma was derived. By essentially non-degraded is meant that for any selected plasma protein, its activity in the concentrated plasma is not less than about 80%, not less than about 85%, not less than about 90%, not less than about 95%, or not less than about 99% of its activity in the starting plasma. In exemplary embodiments, this is true of at least two selected plasma proteins, at least five selected proteins or at least ten selected proteins. In an exemplary embodiment, the overall plasma protein activity of the concentrated plasma is essentially non-degraded when compared to the starting plasma.

In an exemplary embodiment, the concentrated plasma of the invention includes essentially no greater fraction of plasma protein aggregates than were present in the starting plasma. By essentially no greater fraction of plasma protein aggregates is meant, not more than about 2%, not more than about 5%, not more than about 10%, not more than about 15% or not more than about 20% more aggregates on a wt % basis. The wt % basis is calculated from total protein weight in the concentrated plasma pool and starting plasma, i.e., not more than about X % of the plasma protein in the concentrated plasma pool is present as aggregates.

In an exemplary embodiment, the plasma protein fraction isolated according to the method of the invention has characteristics substantially identical to those of the same fractions isolated in the same manner from plasma that has not been concentrated (e.g., frozen plasma) using art-recognized methods. In various embodiments, the characteristics of the plasma protein fraction vary from those of the same protein fractions isolated in the same manner from non-concentrated plasma using art-recognized methods. In a preferred embodiment, the characteristic(s) varying between the two plasma protein fractions correspond(s) to one or more parameter of regulatory relevance, necessary for marketing approval of a therapeutic plasma protein, and the characteristic varies within a range of such one or more parameter by an amount considered insignificant with respect to relevant regulatory requirements for that fraction, i.e., a pharmaceutical formulation incorporating a plasma fraction or a protein isolated from a plasma fraction downstream from the concentrated plasma does not require new regulatory consideration or marketing approval. In various embodiments, the plasma fraction or protein isolated downstream from the concentrated plasma is essentially identical to the corresponding plasma fraction or protein isolated from non-concentrated plasma.

In an exemplary embodiment, the concentrated plasma pool is a starting input for an improved fractionation process. In various embodiments, the concentrated plasma pool improves the process by facilitating and/or promoting one or more of: (i) decreasing fractionation time, (ii) decreasing fractionating material outlay, (iii) decreasing waste, the use of pollutants, e.g., of VOCs, and (iv) increasing throughput with existing fractionating plant infrastructure. In an exemplary embodiment, these results are achieved with essentially no reduction in yield of a selected plasma protein fraction. By essentially no reduction in yield in this instance is meant, when compared to an identical fractionation process commencing with a non-concentrated plasma input, the overall yield of plasma protein is not less than about 2%, not less than about 5%, not less than about 10%, not less than about 15% or not less than about 20% of the overall yield of plasma protein from the process commencing with the non-concentrated input.

In various embodiments, the processing of the physiologically active concentrated plasma is conducted with the addition of one or more component used in plasma fractionation, e.g., alcohol, acid, base. In an exemplary embodiment, the physiologically active concentrated plasma is maintained or passes through a component of a fractionation system, and is incorporated into a process using such a system. In an exemplary embodiment, the fractionation system is a Cohn fractionation system, or a known modification of this system.

In an exemplary embodiment, the invention provides one or more plasma protein fractions, product(s) of a plasma fractionation process commencing with the physiologically active concentrated plasma. In an exemplary embodiment, the plasma protein fraction is a Cohn fraction as this term is understood in the art.

In various embodiments, the invention provides one, two, three, four, five or more unique plasma fraction composition(s) downstream from a physiologically active concentrated plasma input. In various embodiments, the composition is Fraction I paste and comprises fibrinogen, or Fraction I supernatant. In various embodiments, the composition is Fraction II+III (or Fr. I+II+III) paste and comprises IgG, or Fraction II+III (or Fraction I+II+III) supernatant. In some embodiments, the composition is Fraction IV-1 paste and comprises A1PI and/or AT-III, or Fraction IV-1 supernatant. In an exemplary embodiment, the plasma fraction composition is Fraction IV-4 paste and/or Fraction IV-4 supernatant. In various embodiments, the plasma fraction composition is Fraction V paste and comprises albumin, or Fraction V supernatant. In an exemplary embodiment, the fraction or fractions is/are one or more Cohn fraction.

In an exemplary embodiment, the invention provides a preparation of a protease inhibitor prepared by a method of the invention. In various embodiments, the protease inhibitor is selected from alpha 1-antitrypsin, C1-inhibitor, etc.) and a combination thereof.

In an exemplary embodiment, the invention provides a preparation of albumin prepared by a method of the invention.

In an exemplary embodiment, the method provides an aqueous albumin solution containing at least about 5% or at least about 25% by volume of albumin and is suitable for intravenous injection in a human subject, which solution remains stable without precipitation of the albumin after exposure to a temperature of about 45° C. for a period of one month. In an exemplary embodiment, this solution is isolated by fractionation of physiologically active concentrated human plasma of the invention.

In an exemplary embodiment, the invention provides a preparation of IgG isolated from the physiologically active concentrated human plasma. The preparation comprises the IgG in an amount of not less than 60%, 65%, 70%, 75%, 80%, 85%, 90%, or 95% of the amount found in an identical preparation in which IgG is isolated from non-concentrated plasma (e.g., fresh frozen plasma). In various embodiments, the activity of the IgG is not less than 60%, 65%, 70%, 75%, 80%, 85%, 90%, or 95% of the activity of the IgG isolated from non-concentrated plasma (e.g., fresh frozen plasma). The IgG can be polyvalent or hyperimmune IgG.

In an exemplary embodiment, the invention provides a plasma protein isolated from Fraction IV-1 of the fractionated physiologically active concentrated human plasma selected from A1PI, AT-III and a combination thereof. In an exemplary embodiment, the plasma protein is isolated in a yield of not less than about 60%, about 65%, about 70%, about 75%, about 80%, about 85%, about 90%, or about 95% of the yield in which this protein is isolated from non-concentrated plasma (e.g., fresh frozen plasma). In various embodiments, the protein isolated from the physiologically active concentrated human plasma in Fraction IV-1 has an activity of not less than about 60%, about 65%, about 70%, about 75%, about 80%, about 85%, about 90%, or about 95% of the activity of the protein isolated from non-concentrated plasma (e.g., fresh frozen plasma).

In some embodiments, the invention provides a method wherein albumin isolated from Fraction V of the physiologically active concentrated human plasma is isolated in a yield of not less than about 80% of the yield in which this protein is isolated from a non-concentrated plasma input, e.g., fresh frozen plasma. In various embodiments, the albumin isolated from the physiologically active concentrated human plasma has an activity of not less than about 60%, about 65%, about 70%, about 75%, about 80%, about 85%, about 90%, or about 95% of the activity of albumin isolated from non-concentrated plasma (e.g., fresh frozen plasma).

In various embodiments, the invention provides a pharmaceutical formulation comprising one of the plasma protein fractions produced by a method of the invention, or a protein component of one or more such fraction further purified from such fraction. Various pharmaceutical formulations also include a pharmaceutically acceptable vehicle in which the plasma fraction or proteins in/from the fraction (or downstream where further purified) arc formulated.

In various embodiments, the invention provides a pharmaceutical formulation of the invention packaged in a device for administering the pharmaceutical formulation to a subject in need of such administration, e.g., a syringe, infusion bag, and the like. In various embodiments, the device contains a unit dosage formulation of the active protein for administration to a subject in need of such administration. In an exemplary embodiment, the unit dosage is an art-recognized unit dosage for a subject.

### B. Methods

In various embodiments, the present invention provides a novel method of plasma fractionation commencing with a physiologically active concentrated plasma of the invention as the input. An exemplary method of the invention includes providing a physiologically active concentrated plasma solution prepared by, in an exemplary embodiment, ultrafiltration; and submitting the physiologically active concentrated plasma to one or more fractionation process. is Cohn fractionation (FIG. 1), and variations thereof.

In an exemplary embodiment, the starting plasma of use in the methods of the present invention is concentrated after pooling (Cryo-poor, Coagulation Factors-poor, Inhibitors-poor plasma pool). Concentration of the Cohn pool can be achieved by a number of techniques including, but not limited to tangential flow filtration, ultrafiltration and a combination thereof. For example, methods of concentrating the starting plasma include: (a) pre-filtration followed by batch TFF with UF cassettes or (b) pre-filtration followed by single-pass TFF with UF cassettes or (c) UF hollow fiber system with or without pre-filtration.

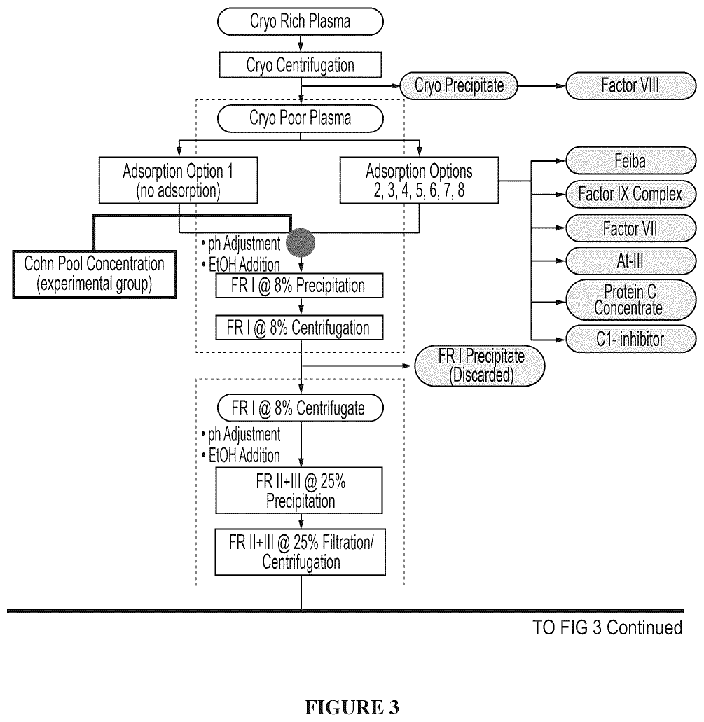

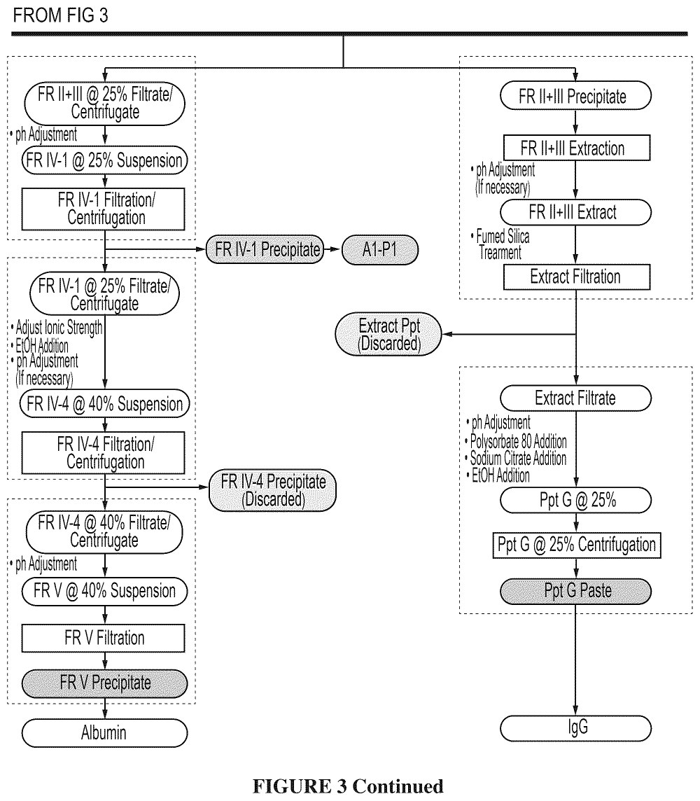

FIG. 3 illustrates an exemplary improved fractionation process of the invention with the filtration step identified and located with a red dot.

The physiologically active concentrated Cohn Pool is, in one embodiment, concentrated through ultrafiltration. The ultrafiltration can be performed in any useful format (i.e., order of addition, temperature, dilution, etc.).

Proteins potentially undergo physical degradation by a number of mechanisms (e.g., clipping, oxidation, unfolding, aggregation, insoluble particulate formation). Many proteins are structurally unstable in solution and are susceptible to conformational changes due to various stresses encountered during purification, processing and storage. These stresses include temperature shift, exposure to pH changes and extreme pH, shear stress, surface adsorption/interface stress, and so on.

As will be appreciated by those of skill in the art, any of these modes of Cohn Pool concentration can be performed singly or in any combination or order.

In various embodiments, the physiologically active concentrated Cohn Pool is composed of at least about 65 g/L plasma protein.

In various embodiments, following cryoprecipitation, the plasma is separated into cryoprecipitate and cryosupernatant. The cryosupernatant or cryosupernatant after adsorption is optionally submitted to further fractionation steps. The separation may be accomplished in any useful fashion, such as, without limitation, centrifugation, filtration or a combination thereof.

In those embodiments in which cooling of the physiologically active concentrated plasma is desired, any useful means of cooling can be utilized. In various embodiments, a vessel or line containing the concentrated plasma is jacketed with a cooling device. In exemplary embodiments, the cooling and/or plasma solution is retained in a vessel, e.g., a jacketed vessel, and, in some embodiments, the plasma solution is cooled during inline flow (“radiator method”).

In some embodiments, the physiological concentrated plasma contains albumin in an amount from about 3.5 to about 5.5 g/dL. In various embodiments, the albumin concentration of the physiologically active concentrated plasma is from about 40% to about 70%, e.g., from about 50% to about 60% of the total plasma protein content of the physiologically active concentrated plasma.

In various embodiments, the albumin in the physiologically active concentrated plasma retains at least about 80%, 85%, 90%, or at least about 95% of the activity on a per unit basis of albumin in plasma.

In some embodiments, the physiological concentrated plasma contains A1PI in an amount from about 50-300 mg/dL, e.g., from about 100 to about 200 mg/dL.

In various embodiments, the A1PI in the physiologically active concentrated plasma retains at least about 80%, 85%, 90%, or at least about 95% of the activity on a per unit basis of A1PI in plasma.

In various embodiments, the physiological concentrated plasma contains IgG in an amount of from about 500 to about 1600 mg/dL, e.g., from about 700 to about 1500 mg/dL.

In various embodiments, the IgG in the physiologically active concentrated plasma retains at least about 80%, 85%, 90%, or at least about 95% of the activity on a per unit basis of IgG in plasma.

In some embodiments, the physiologically active concentrated plasma has an average particle size of about 30 microns or less. In some embodiments, the physiologically active concentrated plasma has a maximum particle size of about 100 microns or less.

In some embodiments, the physiologically active concentrated plasma includes at least 30% plasma protein by weight.

In some embodiments, the physiologically active concentrated plasma is sterile.

In an exemplary embodiment, the invention provides a method of fractionating physiologically active concentrated human plasma using the Cohn fractionation procedure, for example, that procedure set forth in U.S. Pat. No. 2,390,074, wherein the instant improvement comprises the use of physiologically active concentrated human plasma as the starting material for the fractionation procedure. FIG. 1 provides an exemplary process diagram for a method of Cohn fractionation.

Thus, for example, the physiologically active concentrated plasma is submitted to a method of fractionating proteins. An exemplary method involves precipitating a selected protein fraction from a solution containing a plurality of protein fractions. The solution is adjusted to have a pH above the iso-electric point one or more protein in the fraction desired to be precipitated. In an exemplary embodiment, the pH of the fractionation solution is lowered to bring the solution same to approximately the iso-electric point of the desired fraction to be precipitated. An exemplary method comprises bringing the ionic strength of the solution to between 0.1 and 0.2. Various methods include lowering the temperature of the solution to between approximately 0° C. and the freezing point of the solution. In some embodiments an organic precipitant for the plasma protein fraction is added to the protein solution, the amount of the precipitant added being such as to cause precipitation of the desired fraction from the protein solution the said temperature, and separating the precipitate from the solution. In a preferred embodiment, the conditions are adjusted such that substantially only the desired plasma protein fraction precipitates from the solution.

In various embodiments, there is provided a method of fractionating proteins by precipitation from a solution of physiologically active concentrated human plasma containing a plurality of protein fractions, comprises bringing the pH of the solution to approximately the iso-electric point of the desired protein fraction to be precipitated, bring the ionic strength of the solution to between 0.01 and 0.2, lowering the temperature of the solution to between approximately 0° C., and the freezing point of the solution, adding and organic precipitant for protein to the protein solution, the amount of the precipitant added, the pH, the ionic strength and the temperature being such as to cause precipitation of only the desired fraction from the protein solution, and separating the precipitate from the solution.

In various embodiments, in the method for fractionating proteins from a solution of physiologically active concentrated human plasma, the steps which comprise mixing with a solution of proteins an organic precipitant for protein, adjusting the temperature between 0 and −15° C., the amount of the precipitant from about 8% to about 40%, the pH from about 4.4 to about 7 and the ionic strength from about 0.05 to about 0.2, and separating from the resulting liquid system a protein precipitated which is insoluble therein.

In some embodiments, in the method for fractionating proteins from a solution of physiologically active concentrated human plasma, the steps which comprise mixing with a solution of proteins an organic precipitant for protein, adjusting and maintaining the temperature above the freezing point thereof but not above 0° C., the amount of the precipitant from about 10% to about 40%, the pH from about 4.4 to about 7 and the ionic strength from about 0.05 to about 0.2, and separating from the resulting liquid system a protein precipitated which is insoluble therein.

In some embodiments, there is provided a method for fractionating proteins from physiologically active concentrated human plasma, the steps which comprise adding to a containing a mixture of proteins, both an electrolyte and an organic precipitant for protein, the electrolyte being added in an amount sufficient to bring the ionic strength from about 0.01 and 0.2, and the precipitant being added in amount such as to cause precipitation of only the desired protein fraction, adjusting and maintaining the pH of the solution from about 4.4 to about 7 and the temperature thereof from about 0 to about −15° C., thereby precipitating a protein from the resulting system.

In an exemplary embodiment, the invention provides a method of purifying and crystallizing albumin from a solution of concentrated human plasma, which comprises dissolving impure albumin in an alcohol solution containing from about 15 to about 40% alcohol, at a pH of from about 5.5 to to about 6.0, an ionic strength of from about 0.05 to about 0.5 and at a temperature of from about 0° C. to about −5° C., and maintaining the solution within the temperature range until albumin crystallizes from the mixture.

In an exemplary embodiment, in a method of fractionating substances (e.g., proteins) having differing solubilities from a solution of concentrated human plasma at a controlled temperature and hydrogen ion concentration, removing the precipitate thus formed and precipitating a plurality of successive fractions of said substances by variation in one or more of the factors.

In various embodiments, the invention provides a method of preventing denaturation of proteins by modifying reagents which would normally result in denaturation, the method comprising adding the reagents to a protein solution of concentrated human plasma by diffusion through a semi-permeable membrane.

In one embodiment, there is provided a method for fractionating proteins from a solution of physiologically active concentrated human plasma comprising contacting the physiologically active concentrated human plasma with an organic precipitant. An exemplary embodiment includes controlling one or more of the amount of the contacting precipitant, the temperature, the hydrogen ion concentration and the ionic strength of the resulting mixture, separating the resulting precipitate from the supernatant, and separating successive protein fractions by varying a plurality of said factors affecting solubility thereof.

In an exemplary embodiment, the organic precipitant is added a temperature of about 0° or less than about 0° C.

In an exemplary embodiment, the organic precipitant is an alcohol. In various embodiments, it is added a temperature of about 0° or less than about 0° C.

In an exemplary embodiment, there is provided a method of fractionating proteins from a solution of physiologically active concentrated human plasma which comprises, precipitating one or a plurality of different protein fractions from the plasma by the physiologically active concentrated human plasma with an organic precipitant (e.g., alcohol) and by varying the temperature of the mixture of the physiologically active concentrated human plasma and the organic precipitant, the temperature being progressively lowered and the organic precipitant concentration of the mixture being increased, with the precipitation of successive protein fractions. The temperature and the percentage of alcohol are correlated so that the temperature employed for the precipitation of any given protein fraction is close to but above the freezing point of the mixture at the percentage of alcohol and plasma present therein.

Exemplary organic precipitants include ethanol, acetone, dioxane and combinations thereof.

In an exemplary embodiment, IgG isolated from the physiologically active concentrated human plasma is isolated in a yield of not less than about 60%, 65%, 70%, 75%, 80%, 85%, 90%, or not less than about 95% of the yield in which this protein is isolated from fresh frozen plasma. In various embodiments, the activity of the IgG is not less than about 60%, 65%, 70%, 75%, 80%, 85%, 90%, or not less than about 95% of the activity of the IgG isolated from fresh frozen plasma.

In an exemplary embodiment, a protein isolated from Fraction IV-1 of the fractionated physiologically active concentrated human plasma selected from A1PI, AT-III and a combination thereof is isolated in a yield of not less than about 60%, 65%, 70%, 75%, 80%, 85%, 90%, or not less than about 95% of the yield in which this protein is isolated from fresh frozen plasma. In various embodiments, the protein isolated from the physiologically active concentrated human plasma in Fraction IV-1 has an activity of not less than about 60%, 65%, 70%, 75%, 80%, 85%, 90%, or not less than about 95% of the activity of the protein isolated from fresh frozen plasma.

In some embodiments, the invention provides a method wherein albumin isolated from Fraction V of the physiologically active concentrated human plasma is isolated in a yield of not less than about 80% of the yield in which this protein is isolated from fresh frozen plasma. In various embodiments, the albumin isolated from the physiologically active concentrated human plasma has an activity of not less than about 60%, 65%, 70%, 75%, 80%, 85%, 90%, or not less than about 95% of the activity of albumin isolated from fresh frozen plasma.

The methods provided herein allow for the preparation of A1PI compositions having very high levels of purity. For example, in one embodiment, at least about 95% of the total protein in an A1PI composition provided herein is A1PI. In other embodiments, at least about 96% of the protein in this composition is A1PI, or at least about 97%, 98%, 99%, 99.5%, or more of the total protein of the composition is A1PI.

Similarly, the methods provided herein allow for the preparation of A1PI compositions containing extremely low levels of contaminating agents. For example, in certain embodiments, A1PI compositions are provided that contain less than about 10 mg/L contaminant. In other embodiments, the A1PI composition will contain less than about 5 mg/L contaminant, preferably less than about 3 mg/L contaminant, most preferably less than about 2 mg/L contaminant.

In various embodiments, the A1PI in the physiologically active concentrated plasma retains at least about 80%, 85%, 90%, or at least about 95% of the activity on a per unit basis of A1PI in plasma.

In one embodiment, the present invention provides aqueous IgG compositions comprising a protein concentration of from about 150 g/L and about 250 g/L. In certain embodiments, the protein concentration of the IgG composition is from about 175 g/L and about 225 g/L, or from about 200 g/L and about 225 g/L, or any suitable concentration within these ranges, for example at or about, 150 g/L, 155 g/L, 160 g/L, 165 g/L, 170 g/L, 175 g/L, 180 g/L, 185 g/L, 190 g/L, 195 g/L, 200 g/L, 205 g/L, 210 g/L, 215 g/L, 220 g/L, 225 g/L, 230 g/L, 235 g/L, 240 g/L, 245 g/L, 250 g/L, or higher. In a preferred embodiment, the aqueous IgG composition comprises a protein concentration of at or about 200 g/L. In a particularly preferred embodiment, the aqueous IgG composition comprises a protein concentration of at or about 204 g/L.

The methods provided herein allow for the preparation of IgG compositions having very high levels of purity. For example, in one embodiment, at least about 95% of the total protein in an IgG composition provided herein will be IgG. In other embodiments, at least about 96% of the protein is IgG, or at least about 97%, 98%, 99%, 99.5%, or more of the total protein of the composition will be IgG.

Similarly, the methods provided herein allow for the preparation of IgG compositions containing extremely low levels of contaminating agents. For example, in certain embodiments, IgG compositions are provided that contain less than about 100 mg/L IgA. In other embodiments, the IgG composition will contain less than about 50 mg/L IgA, preferably less than about 35 mg/L IgA, most preferably less than about 20 mg/L IgA. In an exemplary embodiment, the IgG preparation contains less than or equal to about 0.14 mg/ml IgA.

In some embodiments, the invention provides a preparation of polyvalent and/or hyperimmune immunoglobulins (IgGs) prepared by a method of the invention. In various embodiments, the IgG is selected from anti-RhO hyperimmune immunoglobulin, anti-hepatitis B hyperimmune immunoglobulin, anti-rabies hyperimmune immunoglobulin, anti-tetanus IgG hyperimmune immunoglobulin and a combination of any two or more thereof.

In various embodiments, the IgG in the physiologically active concentrated plasma retains at least about 80%, 85%, 90%, or at least about 95% of the activity on a per unit basis of IgG in plasma.

### Cohn Pool Concentration Process

Concentration of the Cohn Pool can be achieved by a number of techniques including (a) Pre-filtration followed by batch TFF with UF cassettes or (b) pre-filtration followed by single-pass TFF with UF cassettes or (c) UF hollow fiber system with or without pre-filtration.

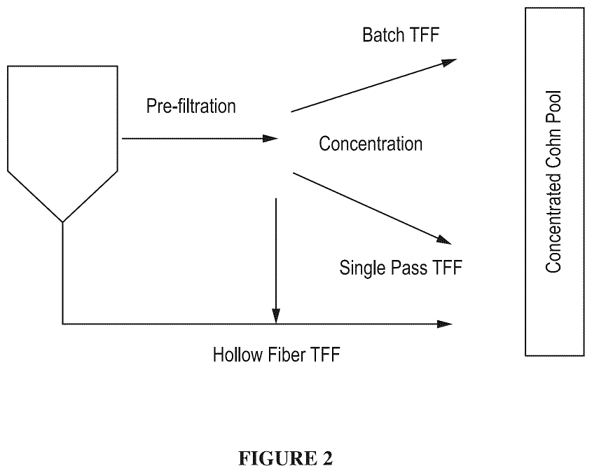

Apparatuses and methods for ultrafiltration are known in art. FIG. 2 provides a process flow diagram of an exemplary method/device of use to concentrate starting Cohn Pool.

An ultrafiltration membrane or ultrafiltration hollow-fiber filter membrane with a nominal molecular weight cut off (NMWCO) of about 300 kDa or less can be used in the concentration of the Cohn Pool, either in re-circulation or in single-pass configuration, and either with or without preceding pre-filtration. In another embodiment, an ultrafiltration membrane or ultrafiltration hollow-fiber filter membrane with a nominal molecular weight cut off (NMWCO) of about 200 kDa or less can be used in the concentration of the Cohn Pool, either in re-circulation or in single-pass configuration, and either with or without preceding pre-filtration. In some embodiment, an ultrafiltration membrane or ultrafiltration hollow-fiber filter membrane with a nominal molecular weight cut off (NMWCO) of about 100 kDa or less can be used in the concentration of the Cohn Pool, either in re-circulation or in single-pass configuration, and either with or without preceding pre-filtration.

The following Examples are offered to illustrate exemplary embodiments of the invention and do not define or limit its scope.

### EXAMPLES

### Example 1

4 study runs were performed until Precipitate G (PptG) and Fraction V (Fr V), respectively. The desired Cohn Pool plasma volume and protein concentration were obtained without significant loss of various plasma derived proteins from different fractionations of Cohn Pool plasma.

For each pair of study runs, i.e., Run 1 and Control 1, Run 2 and Control 2, etc. the same Cohn Pool Batch has been used as starting material. Prior to Cohn fractionation process, approx. 5-12 liters cryo-poor plasma were concentrated in 4 test runs (Run 1, Run 2, Run 3, Run 4) using an ultrafiltration membrane while no ultrafiltration was performed in the 4 respective control runs. For the plasma concentration step, various pre-filters and various TFF filters with a nominal molecular weight cut off (NMWCO) of 100 kDa or less were employed. As a result, the starting protein concentrations of the Cohn Pool plasma in the test runs were 10%-27% higher than that in control run. The study run configuration is shown in Table.

TABLE
Study Run Configuration Overview

| Description | Run 1 | Run 2 | Run 3 | Run 4 |
|---|---|---|---|---|
| Concentration | 27% | 25% | 17% | 10% |
| Factor | | | | |
| Pre-Filter | 1.0 μm PP/ | 1.0 μm PP/ | 0.65 μm GF | 1.0 μm PP/ |
| | 0.45 μm Nylon | 0.45 μm Nylon | | 0.45 μm Nylon |
| TFF | Recirculation | Single Pass | Recirculation | Recirculation |
| Configuration | | | | |
| TFF MWCO | 100 kDa | 30 kDa | 30 kDa | 100 kDa |
| TFF Type | PES | PES | Cellulose | PES |
| | | | Acetate | |

For each protein of interest comparative testing was performed in both concentrated test and non-concentrated control run, respectively. All protein contents in the upstream IgG process until Precipitate G (PptG) in the test runs are overall comparable to the control run as shown in Table-Table.

TABLE
Protein step yields - Cohn Pool

| | | Control 1 | Test 1 | Control 2 | Test 2 | Control 3 | Test 3 | Control 4 | Test 4 |
|---|---|---|---|---|---|---|---|---|---|
| Eq. CPP | L | 6.0 | 6.0 | 6.0 | 6.31 | 6.0 | 6.2 | 6.0 | 6.0 |
| TP by Biuret | g/dL Eq | 5.5 | 5.5 | 5.2 | 5.1 | 5.76 | 5.71 | 5.72 | 5.54 |
| | CPP | | | | | | | | |
| IgG | | 5.62 | 5.61 | 5.0 | 5.0 | 6.10 | 6.10 | 5.5 | 5.3 |
| Albumin | | 32.2 | 32.3 | 31.1 | 29.6 | 32.3 | 33.2 | 33.5 | 33.0 |
| AAT | | 1.29 | 1.23 | 1.23 | 1.17 | 1.30 | 1.29 | 1.26 | 1.24 |
| AMG | g/L Eq | 1.09 | 1.03 | 1.05 | 1.03 | 1.07 | 1.11 | 1.07 | 1.06 |
| | CPP | | | | | | | | |
| HPT | | 1.03 | 0.98 | 1.00 | 0.98 | 1.06 | 1.09 | 1.08 | 1.06 |
| AAG | | 0.72 | 0.69 | 0.70 | 0.68 | 0.72 | 0.72 | 0.73 | 0.70 |
| CER | | 0.21 | 0.20 | 0.21 | 0.20 | 0.22 | 0.21 | 0.20 | 0.21 |
| Fibrinogen | | 2.29 | 2.11 | 1.96 | 1.98 | 2.94 | 3.01 | 2.12 | 2.33 |
| Fibrinogen | μg/mL | 1976 | 1892 | 1802 | 1949 | 2554 | 2635 | 1853 | 1895 |
| | @5% TP | | | | | | | | |

TABLE
Protein step yields - Fraction I Centrifugate

| | | Control 1 | Test 1 | Control 2 | Test 2 | Control 3 | Test 3 | Control 4 | Test 4 |
|---|---|---|---|---|---|---|---|---|---|
| Eq. CPP | L | 6.0 | 6.0 | 6.0 | 6.31 | 6.0 | 6.2 | 6.0 | 6.0 |
| TP by Biuret | g/dL Eq CPP | 5.80 | 5.58 | 5.44 | 5.07 | 5.50 | 5.48 | 5.45 | 5.23 |
| IgG | | 5.58 | 5.33 | 5.1 | 4.9 | 5.81 | 5.86 | 5.52 | 5.38 |
| Albumin | | 32.0 | 30.9 | 30.6 | 29.6 | 32.3 | 32.2 | 32.6 | 32.8 |
| IgA | | 1.50 | 1.42 | 1.46 | 1.41 | 1.55 | 1.56 | 1.59 | 1.54 |
| IgM | | 0.52 | 0.45 | 0.44 | 0.43 | 0.55 | 0.53 | 0.59 | 0.51 |
| C3 | | 1.06 | 0.98 | 1.03 | 0.96 | 1.17 | 1.12 | 1.11 | 1.09 |
| TRF | g/L Eq CPP | 2.11 | 2.01 | 2.08 | 2.01 | 1.98 | 2.00 | 2.16 | 2.10 |
| AAT | | 1.24 | 1.19 | 1.20 | 1.19 | 1.28 | 1.21 | 1.23 | 1.21 |
| AMG | | 1.06 | 1.01 | 1.03 | 1.03 | 1.05 | 1.07 | 1.09 | 1.02 |
| HPT | | 1.00 | 0.97 | 0.97 | 0.95 | 1.04 | 1.04 | 1.06 | 1.02 |
| AAG | | 0.70 | 0.68 | 0.69 | 0.68 | 0.69 | 0.69 | 0.72 | 0.71 |
| CER | | 0.20 | 0.18 | 0.20 | 0.19 | 0.21 | 0.21 | 0.20 | 0.21 |
| Fibrinogen | μg/mL @5% TP | 314 | 300 | 359 | 316 | 377 | 369 | 355 | 312 |

TABLE
Protein step yield - Fraction II + III Filtrate

| | | Control 1 | Test 1 | Control 2 | Test 2 | Control 3 | Test 3 | Control 4 | Test 4 |
|---|---|---|---|---|---|---|---|---|---|
| Eq. CPP | L | 6.0 | 6.0 | 6.0 | 6.31 | 6.0 | 6.2 | 6.0 | 6.0 |
| TP by Biuret | g/dL Eq | 3.60 | 3.23 | 3.51 | 3.25 | 3.49 | 3.41 | 3.43 | 3.70 |
| | CPP | | | | | | | | |
| IgG | | 0.12 | 0.08 | 0.09 | 0.08 | 0.100 | 0.104 | 0.13 | 0.09 |
| Albumin | | 28.4 | 26.0 | 28.0 | 25.4 | 27.6 | 27.3 | 29.9 | 29.4 |
| IgA | | 0.119 | 0.070 | 0.030 | 0.057 | 0.037 | 0.067 | 0.238 | 0.085 |
| IgM | | 0.010 | 0.007 | 0.010 | 0.007 | 0.010 | 0.008 | 0.010 | 0.009 |
| C3 | | 0.011 | 0.007 | 0.011 | 0.007 | 0.010 | 0.009 | 0.010 | 0.009 |
| TRF | g/L Eq | 1.75 | 1.57 | 1.77 | 1.63 | 1.67 | 1.69 | 1.75 | 1.73 |
| | CPP | | | | | | | | |
| AAT | | 0.59 | 0.52 | 0.57 | 0.58 | 0.55 | 0.52 | 0.74 | 0.60 |
| AMG | | 0.15 | 0.13 | 0.14 | 0.16 | 0.18 | 0.15 | 0.15 | 0.12 |
| HPT | | 0.87 | 0.78 | 0.82 | 0.79 | 0.91 | 0.93 | 0.89 | 0.90 |
| AAG | | 0.61 | 0.54 | 0.58 | 0.56 | 0.61 | 0.60 | 0.61 | 0.61 |
| CER | | 0.13 | 0.10 | 0.12 | 0.10 | 0.12 | 0.11 | 0.12 | 0.11 |

TABLE
Protein step yields - Fraction II + III Paste Extract CUNO Filtrate

| | | Control 1 | Test 1 | Control 2 | Test 2 | Control 3 | Test 3 | Control 4 | Test 4 |
|---|---|---|---|---|---|---|---|---|---|
| Eq. CPP | L | 6.0 | 6.0 | 6.0 | 6.31 | 6.0 | 6.2 | 6.0 | 6.0 |
| TP by | g/dL Eq CPP | 0.57 | 0.52 | 0.53 | 0.54 | 0.60 | 0.65 | 0.60 | 0.62 |
| Biuret | | | | | | | | | |
| IgG | | 4.51 | 4.02 | 4.04 | 4.03 | 4.67 | 4.82 | 4.52 | 4.54 |
| Albumin | | 0.42 | 0.60 | 0.29 | 0.44 | 0.49 | 0.66 | 0.45 | 0.60 |
| IgA | | 0.775 | 0.708 | 0.731 | 0.775 | 0.831 | 0.910 | 0.742 | 0.815 |
| IgM | g/L Eq CPP | 0.223 | 0.189 | 0.148 | 0.112 | 0.150 | 0.254 | 0.145 | 0.175 |
| C3 | | 0.120 | 0.104 | 0.088 | 0.077 | 0.089 | 0.112 | 0.104 | 0.129 |
| AAT | | 0.05 | 0.07 | 0.02 | 0.04 | 0.04 | 0.11 | 0.02 | 0.05 |
| AMG | | 0.71 | 0.64 | 0.61 | 0.58 | 0.60 | 0.68 | 0.65 | 0.66 |
| Fibrinogen | ug/mL@5% TP | 26 | 41 | 67.5 | 61.6 | 71.5 | 71.6 | 47.4 | 69.8 |

TABLE
Precipitate G Protein Composition

| | | Control 1 | Test 1 | Control 2 | Test 2 | Control 3 | Test 3 | Control 4 | Test 4 |
|---|---|---|---|---|---|---|---|---|---|
| Eq. CPP | L | 6.0 | 6.0 | 6.0 | 6.31 | 6.0 | 6.2 | 6.0 | 6.0 |
| TP by Biuret | g/dL | 5.97 | 6.13 | 6.12 | 6.17 | 6.81 | 6.34 | 5.9 | 6.0 |
| IgG | ug/mL@ 5% TP | 4420 | 4200 | 3983 | 3993 | 4017 | 3893 | 4153 | 3795 |
| Albumin | | 13.5 | 13.1 | 10.4 | 11.5 | 14.5 | 14.7 | 11.7 | 15.7 |
| IgA | | 663 | 642 | 628 | 667 | 607 | 638 | 594 | 617 |
| IgM | | 134 | 136 | 127 | 122 | 115 | 112 | 128 | 144 |
| C3 | | 74 | 75 | 85 | 73 | 75.6 | 69.8 | 83 | 97 |
| AAT | | 6.08 | 5.98 | 5.13 | 6.79 | 8.40 | 9.93 | 4.62 | 10.64 |
| AMG | | 398 | 390 | 345 | 345 | 282 | 321 | 311 | 336 |
| Fibrinogen | ug/mL@ 5% TP | 29.2 | 28.3 | 61.8 | 60.3 | 77.0 | 76.2 | 47.2 | 75.4 |
| FXI | E/ml @5% TP | 0.017 | 0.024 | 0.025 | 0.024 | 0.022 | 0.024 | 0.02 | 0.1 |
| FXII | mU/ml @5% | 4.0 | 4.3 | 11.2 | 12.7 | 6.9 | 11.7 | 4.9 | 5.9 |
| | TP | | | | | | | | |
| PL-1 | nmol/mL min | 13.1 | 8.2 | 15.1 | 19.2 | 18.2 | 14.8 | 12.6 | 15.2 |
| | @5% TP | | | | | | | | |
| PKA | IE/mL @5% | 11.1 | 7.2 | 37 | 49 | 21 | 8.5 | 15.0 | 26.5 |
| | TP | | | | | | | | |
| KKA | nmol/mL min | 119.0 | 75.9 | 118 | 133 | 106 | 80 | 96.3 | 122.3 |
| | @ 5% TP | | | | | | | | |

Proteins typically found in the Fraction IV and V downstream from Cohn pool concentration are found in these fractions in yields and purity comparable to those found in Fraction IV and V in a process starting with non-concentrated Cohn Pool. The conditions of exemplary separation and purification processes for those plasma derived product intermediates produced from Cohn Pool concentrate are set forth in

### Table—

[symbol]

[symbol]

indicates data missing or illegible when filed

### Table.

[symbol]

[symbol]

indicates data missing or illegible when filed

The supernatant of Fraction II+III was further submitted to fractionation procedure. The supernatant of Fraction II+III was contacted with 25% Ethanol to obtain Fraction IV-1 precipitate.

TABLE
Protein step yields - Fraction IV-1 Filtrate

| | | Control 1 | Test 1 | Control 2 | Test 2 | Control 3 | Test 3 | Control 4 | Test 4 |
|---|---|---|---|---|---|---|---|---|---|
| Eq. CPP | L | 6.0 | 6.0 | 6.0 | 6.31 | 6.0 | 6.2 | 6.0 | 6.0 |
| TP by | g/dL Eq | 2.80 | 2.39 | 2.94 | 2.73 | 2.70 | 2.62 | 2.70 | 2.70 |
| Biuret | CPP | | | | | | | | |
| Albumin | | 27.1 | 23.6 | 26.1 | 23.9 | 25.2 | 24.6 | 27.3 | 25.8 |
| TRF | | 0.88 | 0.77 | 1.08 | 1.05 | 0.64 | 0.64 | 0.96 | 0.78 |
| AAT | | 0.039 | 0.029 | 0.015 | 0.017 | 0.016 | 0.019 | 0.060 | 0.036 |
| HPT | | 0.59 | 0.50 | 0.58 | 0.57 | 0.56 | 0.53 | 0.66 | 0.59 |
| AAG | | 0.59 | 0.50 | 0.58 | 0.52 | 0.54 | 0.53 | 0.60 | 0.56 |
| PKA | | 7.9 | 20.1 | 222 | 330 | 57 | 8 | 59.0 | 29.9 |
| KKA | | 27.8 | 15.3 | 441 | 604 | 201 | 79 | 185.1 | 135.6 |

TABLE
Protein Step yields - Fraction IV-4 Filtrate

| | | Control 1 | Test 1 | Control 2 | Test 2 | Control 3 | Test 3 | Control 4 | Test 4 |
|---|---|---|---|---|---|---|---|---|---|
| Eq. CPP | L | 6.0 | 6.0 | 6.0 | 6.31 | 6.0 | 6.2 | 6.0 | 6.0 |
| TP by Biuret | g/dL Eq CPP | 2.09 | 1.60 | 2.08 | 1.70 | 2.09 | 1.97 | 2.09 | 2.03 |
| Albumin | | 22.3 | 17.9 | 21.1 | 17.1 | 20.6 | 19.7 | 22.0 | 20.7 |
| TRF | | 0.03 | 0.02 | 0.03 | 0.016 | | | | |
| AAT | | 0.01 | 0.01 | 0.01 | 0.008 | | | | |
| HPT | | 0.05 | 0.02 | 0.23 | 0.30 | 0.08 | 0.06 | 0.10 | 0.10 |
| AAG | | 0.56 | 0.45 | 0.50 | 0.40 | 0.50 | 0.49 | 0.53 | 0.50 |
| CER | | 0.007 | 0.005 | 0.006 | 0.004 | | | | |

TABLE
Fraction V Protein Composition

| | | Control | Test | Control | Test | Control | Test | Control | Test |
|---|---|---|---|---|---|---|---|---|---|
| | | 1 | 1 | 2 | 2 | 3 | 3 | 4 | 4 |
| Eq. CPP | L | 6.0 | 6.0 | 6.0 | 6.31 | 6.0 | 6.2 | 6.0 | 6.0 |
| TP by Biuret | g/dL | 6.04 | 6.19 | 6.05 | 6.19 | 5.97 | 6.01 | 5.7 | 6.1 |
| Albumin | ug/mL@ 5% TP | 5548 | 5601 | 4509 | 4434 | 5212 | 5263 | 5306 | 5319 |
| HPT | | 27.5 | 23.4 | 32.5 | 29.4 | 35.0 | 28.4 | 5306 | 5319 |
| TRF | | 1.5 | 1.4 | | | | | 35 | 33 |
| AAT | | 0.7 | 0.7 | 0.74 | 0.73 | | | | |
| AAG | | 10.2 | 11.7 | 10.1 | 11.6 | 8.8 | 11.5 | 10.2 | 11.2 |
| CER | | 0.38 | 0.37 | | | | | | |
| PKA | IE/mL @5% TP | 3.3 | 4.0 | 8.3 | 18.5 | 23 | 7.9 | 14.3 | 16.8 |
| KKA | nmol/mL min @ | 8.3 | 8.9 | 68 | 107 | 49 | 21 | 24.4 | 23.9 |
| | 5% TP | | | | | | | | |

### Example 2

Cohn Pool Concentration (CPC) aims to increase capacity and reduce operational costs with minimal disruption to commercial supply. The process of present invention makes feasible of concentrating Cohn Pool plasma through ultrafiltration. Ultrafiltration permits the selective separation, concentration, and purification of protein components. Pilot scale runs have been performed in present invention and the desired Cohn Pool plasma volume and protein concentration were obtained without significant loss of various plasma derived proteins from different fractionations of Cohn Pool plasma.

Prior to Cohn fractionation process, about 100 liters cryo-poor plasma were concentrated in test runs (Run 1, Run 2, Run 3) using an ultrafiltration membrane while no ultrafiltration was performed in the control run. For the plasma concentration step, hollow-fiber filters with a nominal molecular weight cut off (NMWCO) of 100 kDa or less was employed. As a result, the starting protein concentrations of the Cohn Pool plasma in the test runs were about 15% higher than that in control run. For each protein of interest, technical specification of the separate and purified protein, for example, recovery rate, quality, are tabulated and analyzed.

### 1. Immunoglobulin Recovery Rate and Quality Were Not Affected by Cohn Pool concentration

Immunoglobulin content in all runs was calculated following the concentration step. The resulting IgG content in the Cohn Pool plasma prepared for Ethanol treatment is concentrated without significant loss. The Upstream IgG Efficiency in the test runs are comparable to the control run as shown in Table 1.

The recovery rate of purified IgG from Precipitate G (PptG) was also compared between test runs and control. Fraction II+III precipitate resulting from Cohn fractionation process were suspended in a suspension buffer, thereby forming an IgG suspension. The IgG suspension were filtered, and the filtrate were treated with detergent. After adjusting the pH of detergent-treated filtrate to about 7.0 and adding ethanol to a final concentration of from about 20% to about 30%, Precipitate G was formed. Table 2 shows that the IgG purification efficiency was not affected by the process of CPC. Comparable IgG yields from PptG between test runs and the non-concentrated control run have been observed in the pilot scale study. IgG quality was evaluated at drug product level for all runs. All results met all limits as shown in Table 3.

TABLE 1
Immunoglobulin balance in upstream processing

| | | | | Delta % | | |
|---|---|---|---|---|---|---|
| | | | | (Run 1 vs. | | |
| | | | Run 1 | Run 1 | | |
| Parameter/Step | Unit | Run 1 | Control¹ | Control) | Run 2 | Run 3 |
| Starting Material | N/A | | Cohn | | Cohn | Cohn |
| | | | Plasma | | Plasma | Plasma |
| | | | after Cryo | | after | after |
| | | | separation | | adsorption: | adsorption: |
| | | | | | FEIBA², | Factor IX, |
| | | | | | AT III³ | Factor VII |
| Cohn Plasma (Starting | [L] | 100.5 | 100.5 | | 112.7 | 120.3 |
| Volume) | | | | | | |
| Cohn Plasma for EtOH | [L] | 86.4 | 100 | | 95.7 | 102.0 |
| Frac | | | | | | |
| Total Protein (pool | [g/L] | 58.9 (after | 50.5 | | 53.7 (63.9 | 53.7 (64.8 |
| before conc.) | | conc.) | | | after | after |
| | | | | | conc.) | conc.) |
| IgG (pool before conc.) | [g/L] | 5.71 (after | 4.92 | | 5.07 (6.14 | 5.22 (6.10 |
| | | conc.) | | | after | after |
| | | | | | conc.) | conc.) |
| Total IgG | [g] | 493 | 494 | | 588 | 622 |
| Extract Filtrate | [L] | 98.6 | 120.2 | | 107.8 | 114.2 |
| IgG | [g/L] | 4.67 | 3.71 | | 4.96 | 4.85 |
| Total IgG | [g] | 460.3 | 445.9 | +3.2% | 534.6 | 553.8 |
| IgG g/L plasma before | [g/L] | 4.55 | 4.40 | +3.2% | 4.74 | 4.53 |
| conc | | | | | | |
| Upstream IgG | [%] | 93.1 | 90.2 | +3.2% | 93.5 | 88.2 |
| Efficiency | | | | | | |

¹Same starting material was used for ‘Run 1 Control’ and ‘Run 1’, i.e., Cohn Plasma after Cryo separation was split and processed without Cohn Pool Concentration (‘Run 1 Control’) and with Cohn Pool Concentration (‘Run 1’) in parallel.
²Factor eight inhibitor bypass activity
³Antithrombin III

TABLE 2
Immunoglobulin Balance in Test Runs and Control Run after Purification

| | | | | Delta % | | |
|---|---|---|---|---|---|---|
| | | | Run 1 | (Run 1 vs. | | |
| | | Run 1 | Control | Run 1 | | |
| | Unit | Ppt G | Ppt G | Control) | Run 2 | Run 3 |
| CPP | L | 100.5 | 100.5 | 0.0 | 112.7 | 120.3 |
| PptG weighed in | kg | 1.744 | 1.778 | | 2.343 | 2.199 |
| fractionation | | | | | | |
| Retrieved for | kg | 0.996 | 1.002 | | 1.000 | 1.004 |
| suspension | | | | | | |
| TP in suspension | g | 310.0 | 288.6 | 7.4 | 283.3 | 307.7 |
| IgG in suspension | g | 264.3 | 244.9 | 7.9 | 222.3 | 251.0 |
| IgG per kg of paste | g IgG/kg | 265.3 | 244.4 | 8.5 | 222.2 | 250.0 |
| | PptG | | | | | |
| IgG in paste/CPP Eq | g IgG/L | 4.60 | 4.32 | 6.4 | 4.62 | 4.57 |
| | CPP | | | | | |
| TP loaded on filter | g | 260 | 260 | | 260 | 260 |
| TP loaded on filter | g | 221 | 220 | | 212 | 221 |
| (minus losses) | | | | | | |
| IgG loaded on filter | g | 226.8 | 225.8 | 0.4 | 210.9 | 216.9 |
| IgG loaded on filter | g | 193.9 | 192.1 | 0.9 | 170.7 | 184.0 |
| (minus losses) | | | | | | |
| PptG Eq loaded on | kg | 0.836 | 0.903 | −7.4 | 0.918 | 0.848 |
| filter | | | | | | |
| PptG Eq loaded on | kg | 0.710 | 0.763 | −7.0 | 0.749 | 0.720 |
| filter (minus losses) | | | | | | |
| CPP Eq | L | 48.2 | 51.0 | −5.6 | 44.2 | 46.4 |
| CPP Eq | L | 40.9 | 43.2 | −5.2 | 36.0 | 39.4 |
| (minus losses) | | | | | | |
| IgG After CM | g | 176.8 | 182.5 | | 173.5 | 172.6 |
| IgG CM/Suspension | % | 78.0 | 80.8 | −3.5 | 82.3 | 79.6 |
| Yield | | | | | | |
| IgG CM/Suspension | % | 86.0 | 89.5 | −3.9 | 94.1 | 87.9 |
| Yield (incl losses) | | | | | | |
| Final solution weight | kg | 1.6551 | 1.6756 | | 1.6196 | 1.6699 |
| Recovered IgG | g | 160.4 | 162.4 | −1.2 | 156.6 | 161.6 |
| IG Efficiency | % | 70.7 | 71.9 | −1.7 | 74.3 | 74.5 |
| (Purification | | | | | | |
| Efficiency) | | | | | | |
| IG Efficiency | % | 82.7 | 84.5 | −2.1 | 91.7 | 87.9 |
| (Purification Efficiency | | | | | | |
| incl. losses) | | | | | | |
| Recovered IgG/CPP Eq | g IgG/L | 3.33 | 3.18 | 4.7 | 3.55 | 3.48 |
| | CPP | | | | | |
| Recovered IgG/CPP Eq | g IgG/L | 3.92 | 3.76 | 4.2 | 4.35 | 4.10 |
| (incl losses) | CPP | | | | | |

TABLE 3
Immunoglobulin Drug Product Test Results

| | Acceptance | | | Run 1 | | |
|---|---|---|---|---|---|---|
| Test | Criteria | Units | Run 1 | Control | Run 2 | Run 3 |
| Diphtheria | ≥1.2 U of | U/mL | 10.4 | 9.8 | 9.8 | 12.2 |
| Antibody | US standard | | | | | |
| | Antitoxin/mL | | | | | |
| Poliomyelitis | ≥0.2 | times the | 1.1 | 0.9 | 1.0 | 1.2 |
| Antibody | | antibody | | | | |
| | | level of | | | | |
| | | CBER | | | | |
| | | Reference | | | | |
| | | Polio | | | | |
| | | Immune | | | | |
| | | Globulin | | | | |
| | | Lot 176 | | | | |
| IgA | ≤0.14 | mg/mL | 0.06 | 0.06 | 0.06 | 0.07 |
| IgM | ≤10 | mg/dL | <4 | <4 | <4 | <4 |
| Anti-A | NMT 1/64 | 1/ | 32 | 16 | 16 | 16 |
| | @25 g/L of | | | | | |
| | IgG | | | | | |
| Anti-B | NMT 1/64 | 1/ | 8 | 8 | 16 | 16 |
| | @25 g/L of | | | | | |
| | IgG | | | | | |
| Anti-D | NMT NIBSC | — | Satisfactory | Satisfactory | Satisfactory | Satisfactory |
| antibodies | Ref. 02/228 | | | | | |
| | (or | | | | | |
| | equivalent) | | | | | |
| Anti- | NMT 50 | % | 33 | 38 | 34 | 34 |
| complement | corresponding | | | | | |
| Activity | to 1 CH50 | | | | | |
| | U/mg protein | | | | | |
| Total Protein | 9.0-11.0 | g/dL | 10.0 | 9.9 | 10.0 | 10.0 |
| Appearance | The liquid | — | Satisfactory | Satisfactory | Satisfactory | Satisfactory |
| | preparation is | | | | | |
| | clear or | | | | | |
| | slighlty | | | | | |
| | opalescent | | | | | |
| | and colorless | | | | | |
| | or pale | | | | | |
| | yellow | | | | | |
| Glycine | 0.20-0.30/ | M | 0.23 | 0.23 | 0.23 | 0.24 |
| | 0.21-0.26 | | | | | |
| HAV Antibody | ≥3.5 | IU/mL | 5.8 | 8.2 | 5.9 | 6.0 |
| HBsAg | ≥0.20 | IU/mL | 8.17 | 7.61 | 7.09 | 6.92 |
| Antibody | | | | | | |
| Molecular Size | Monomer + | % | 100 | 100 | 100 | 100 |
| Distribution | Dimer: ≥95 | | | | | |
| | Polymer: ≤2 | % | 0.1 | 0.1 | 0.1 | 0.1 |
| | Fragment: ≤3 | % | 0.3 | 0.2 | 0.2 | 0.2 |
| Purity | ≥98 | % | 100 | 100 | 100 | 100 |
| Osmolality | 240-300 | mOsmol/ | 268 | 269 | 263 | 264 |
| | | kg | | | | |
| Parvo B19 | ≥50 | IU/mL | 414 | 459 | 441 | 367 |
| pH | 4.6-5.1 | — | 4.7 | 4.7 | 4.7 | 4.7 |
| | Diluted @ | | | | | |
| | 1% with | | | | | |
| | 0.9% NaCl | | | | | |
| Prekallikrein | ≤10 | — | <5 | <5 | <5 | <5 |
| Activator | | | | | | |
| Activity | | | | | | |
| Tween 80 | ≤100 | ppm | <26 | <26 | <26 | <26 |
| TNBP | ≤1.0 | ppm | <0.2 | <0.2 | <0.2 | <0.2 |
| Triton X-100 | ≤1.0 | ppm | <0.1 | <0.1 | <0.1 | <0.1 |
| Density | N/A | g/cm³ | 1.03 | 1.03 | 1.03 | 1.03 |
| THP-1/IL-1ra | NLT 75% | % | 115 | 128 | 137 | 126 |
| Release | | | | | | |
| Ig G 1 | N/A | mg/mL | 64.0 | 65.6 | | |
| Subclass | 52.6-68.4 | % | 62.4 | 62.4 | 58.6 | 59.2 |
| Distribution | | | | | | |
| Ig G 2 | N/A | mg/mL | 30.9 | 32.0 | | |
| Subclass | 23.5-40.5 | % | 30.2 | 30.5 | 33.3 | 32.9 |
| Distribution | | | | | | |
| Ig G 3 | N/A | mg/mL | 5.0 | 4.8 | | |
| Subclass | 2.9-7.8 | % | 4.8 | 4.6 | 5.1 | 5.1 |
| Distribution | | | | | | |
| Ig G 4 | N/A | mg/mL | 2.7 | 2.6 | | |
| Subclass | 1.2-3.2 | % | 2.6 | 2.5 | 3.0 | 2.8 |
| Distribution | | | | | | |
| IgE | ≤149.6 | IU/mL | 53.9 | 52.7 | 42.0 | 48.6 |
| Complement- | ≥60 (cfr. EP) | % | 107 | 106 | 91 | 84 |
| mediated Lysis | | | | | | |
| of Red Blood | | | | | | |
| Cells | | | | | | |
| Haemophilus | ≥1:1600 | — | 1:12800 | 1:12800 | 1:12800 | 1:12800 |
| Influenzae Ab | | | | | | |
| Albumin | <0.22 | g/L | <0.22 | <0.22 | <0.222 | <0.222 |
| PL-1 | <10 | nmol/mL × | <10 | <10 | <10 | <10 |
| Amidolytic | | min | | | | |
| activity | | | | | | |
| Fibrinogen | ≤1.5 | μg/mL | <0.2 | <0.2 | <0.2 | <0.2 |
| Plasminogen | ≤0.3 | μg/mL | <0.12 | <0.12 | <0.12 | <0.12 |
| Alcohol | <20 | μg/mL | <20 | <20 | <20 | <20 |
| (Ethanol) | | | | | | |
| Aluminum | ≤34 | μg/L | <25 | <25 | <25 | <25 |
| FXIa | Control | mU/mL | 0.47 | <0.3125 | <0.3125 | <0.3125 |
| | limit: ≤1.6 | | | | | |
| | mU/mL FXIa | | | | | |
| | Action | | | | | |
| | limit: ≤6.0 | | | | | |
| | mU/mL FXIa | | | | | |
| FXIa @5% | Control | mU/mL | <0.3125 | <0.3125 | <0.3125 | <0.3125 |
| | limit: ≤1.6 | | | | | |
| | mU/mL FXIa | | | | | |
| | Action | | | | | |
| | limit: ≤6.0 | | | | | |
| | mU/mL FXIa | | | | | |
| FXI Protein | N/A | U/mL | 0.11 | 0.04 | 0.02 | 0.01 |
| TGA(Thrombin | <132% NP | % NP | 120 | 109 | 110 | 105 |
| Generation | equivalent to | | | | | |
| Assay) | 1.7 mU/mL | | | | | |
| | FXIa | | | | | |

### 2. Process Parameters for Immunoglobulin Separation and Purification From Cohn Pool

The conditions for an exemplary plasma derived immunoglobulin proteins separation process, including Cohn Pool concentration, fraction I precipitation and separation, fraction II+III precipitation and separation, fraction II+III extract precipitation and separation, precipitate G (PptG) precipitation and separation, are set forth in Table 4-8.

In the exemplary CPC process, target amounts of filter aid and filter area were calculated based on the volume of concentrated Cohn Pool instead on the volume of cryo-poor plasma (CPP). Cohn Pool was concentrated of at least about 14% (w/v) by ultrafiltration, forming a first Cohn Pool concentrate. The technical protocol and parameters associated with the process of ultrafiltration in all runs were indicated in Table 4.

TABLE 4
Parameters for Cohn Pool Concentration Step

| Description | Unit | Run 1 | Run 2 | Run 3 |
|---|---|---|---|---|
| Cohn Pool mass | [kg] | 102.6 | 115.1 | 122.8 |
| before concentration | | | | |
| Cohn Pool volume | [L] | 100.5 | 112.7 | 120.3 |
| before concentration | | | | |
| Type of filter for | N/A | hollow- fiber | hollow- fiber | hollow- fiber |
| concentration | | 100 kDa MWCO | 100 kDa MWCO | 100 kDa MWCO |
| Filter surface area | [m²] | 0.7 | 1.8 | 1.8 |
| Number of filters used | N/A | 1 | 1 | 1 |
| Filtration time | [hh:mm] | 05:31 | 02:58 | 02:44 |
| Maximum sustained TMP | [bar] | 0.52 | 0.46 | 0.42 |
| Average retentate flowrate | [L/min] | 1.08 | 1.69 | 1.73 |
| Average permeate flowrate | [g/min] | 50.3 | 110.7 | 119.9 |
| Permeate weight | [kg] | 16.66 | 19.70 | 19.66 |
| Initial concentration | [%] | 16.24% | 15.87% | 16.01% |
| factor (by mass) | | | | |
| Post-wash using 0.9% NaCl | [kg] | 0.415 | 0.579 | 0.576 |
| Cohn Pool mass after | [kg] | 88.2 | 105.6 | 104.4 |
| concentration & post-wash | | | | |
| Cohn Pool volume after | [L] | 86.4 | 95.7 | 102.0 |
| concentration & post-wash | | | | |
| Final concentration | [%] | 14.04% | 14.91% | 14.98% |
| factor (by mass) | | | | |
| Filter load | [L CPP/m²] | 143.6 | 62.6 | 66.8 |
| Turbidity - before conc./ | [NTU] | 223/249 | 262/288 | 270/309 |
| after conc. | | | | |

The first Cohn Pool concentrate was further submitted to fractionation procedure. The first Cohn Pool concentrate was contacted with 8% ethanol at a pH of from about 7.0 to 7.5 to obtain a Fraction I precipitate and a Fraction I supernatant from the fractionated Cohn Pool. The technical protocol and parameters associated with fraction I precipitation and separation in all runs were indicated in Table 5.

TABLE 5
Parameters of Fraction I Precipitation and Separation with 8% Ethanol

| | | | Run 1 | | |
|---|---|---|---|---|---|
| Parameter | Unit | Run 1 | Control | Run 2 | Run 3 |
| pH Before Alcohol | [pH] | 7.21 | 7.19 | 7.01 | 7.0 |
| Temperature Before | [° C.] | 0.2 | 0.0 | 1.1 | 0.9 |
| Alcohol | | | | | |
| Alcohol Add Time | [hh:mm] | 01:00 | 01:01 | 01:00 | 01:00 |
| pH After Alcohol | [pH] | 7.38 | 7.38 | 7.17 | 7.17 |
| Temperature After | [° C.] | −0.8 | −0.5 | −0.4 | −0.3 |
| Alcohol | | | | | |
| Final pH | [pH] | 7.51 → 7.18 | 7.47 → 7.21 | 7.41 | 7.32 |
| Total Aging Time | [hh:mm] | 13:51 | 13:51 | 15:53 | 17:25 |
| Suspension Weight | [kg] | 94.7 | 110.2 | 113.3 | 112.1 |
| Separation Method | N/A | CEPA Z61H | CEPA Z61H | CEPA Z61H | CEPA Z61H |
| Supernatant Density | [kg/L] | 1.008 | 1.006 | 1.010 | 1.010 |
| Total Supernatant | [L] | 92.7 | 108.1 | 103.1 | 109.2 |
| Volume | | | | | |
| Paste Weight | [kg] | 0.709 | 0.704 | 1.081 | 1.047 |
| Duration of | [hh:mm] | 02:03 | 02:21 | 02:36 | 01:20 |
| Centrifugation | | | | | |
| Average Flowrate | [L/h] | 45.2 | 46.0 | 39.7 | 81.9 |

The Fraction I supernatant was further contacted with about 25% ethanol at a pH of from about 6.7 to about 7.3 to form a Fraction II+III precipitate. The technical parameters associated with isolating proteins from fraction II+III of fractionated Cohn Pool in all runs were indicated in Table 6.

TABLE 6
Parameters of Fraction II + III Precipitation and Separation with 25% Ethanol

| | | | Run 1 | | |
|---|---|---|---|---|---|
| Parameter | Unit | Run 1 | Control | Run 2 | Run 3 |
| pH Before Alcohol | [pH] | 6.71 | 6.67 | 6.68 | 6.73 |
| Temperature Before | [° C.] | −0.9 | −0.9 | −1.0 | −1.9 |
| Alcohol | | | | | |
| Alcohol Add Time | [hh:mm] | 03:00 | 03:00 | 03:00 | 03:00 |
| pH After Alcohol | [pH] | 7.18 → 6.92 | 7.05 → 6.89 | 7.08 → 6.89 | 7.20 → 6.89 |
| Temperature After | [° C.] | −6.6 | −6.5 | −6.5 | −6.6 |
| Alcohol | | | | | |
| Total Aging Time | [hh:mm] | 16:31 | 16:25 | 17:11 | 18:14 |
| Final pH | [pH] | 7.09 | 6.96 | 7.34 → 6.90 | 7.33 → 6.91 |
| Suspension Weight | [kg] | 111.6 | 129.9 | 124.4 | 131.7 |
| Separation Method | N/A | Eaton 400 | Eaton 400 | Eaton 400 | Eaton 400 |
| | | filter press | filter press | filter press | filter press |
| Filter Type | N/A | Becopad | Becopad | Becopad | Becopad |
| | | P270 | P270 | P270 | P270 |
| Effective Surface Area | [m²] | 1.21 | 1.32 | 1.32 | 1.43 |
| Filter Load | [L CP/m²] | 83.1 | 76.1 | 85.4 | 84.1 |
| Frame Thickness (single) | [mm] | 25 | 25 | 25 | 25 |
| Frame Volume (single) | [L] | 2.20 | 2.20 | 2.20 | 2.20 |
| Dead volume | [L] | 13.1 | 14.2 | 14.2 | 15.3 |
| Filteraid Type | N/A | Fibracel | Fibracel | Fibracel | Fibracel |
| Filteraid Target | [g/L] | 22 g/L CPC | 22 g/L CPP | 22 g/L CPC | 22 g/L CPC |
| Filteraid Amount | [kg] | 1.901 | 2.200 | 2.105 | 2.243 |
| Pre-Rinse Vol (water) | [L] | 42.4 | 46.2 | 46.2 | 50.1 |
| Pre-Wash Vol (wash | [L] | 59.1 | 63.2 | 68.3 | 62.1 |
| solution) | | | | | |
| Post-Wash Vol (wash | [L] | 13.1 | 14.2 | 14.2 | 15.3 |
| solution) | | | | | |
| Temp (end of pre-wash) | [° C.] | −2.6 | −2.8 | −4.0 | −3.5 |
| Mean Filtration Temp | [° C.] | −4.9 | −5.0 | −5.2 | −5.1 |
| Max Sustained Pressure | [bar] | 1.0 | 0.9 | 1.0 | 1.1 |
| (filtration) | | | | | |
| Final Flux | [L/hr/m²] | 42.70 | 47.87 | 24.91 | 25.0 |
| Supernatant Density | [kg/L] | 0.979 | 0.979 | 0.981 | 0.981 |
| Total Supernatant Volume | [L] | 129.2 | 150.4 | 142.4 | 149.4 |
| Paste Weight | [kg] | 7.822 | 8.58 | 10.07 | 10.512 |
| Duration of Filtration | [hh:mm] | 01:53 | 01:48 | 03:11 | 03:17 |
| Duration of Filtration + | [hh:mm] | 02:30 | 02:23 | 04:20 | 04:19 |
| Post-Wash | | | | | |

The Fraction II+III precipitate was further suspended in a suspension buffer, thereby forming an IgG suspension. After mixing finely divided silicon dioxide (SiO₂) with the IgG suspension for at least about 30 minutes, IgG suspension was filtered and resulted a filtrate and a filter cake. The technical parameters associated with isolating proteins from fraction II+III extract in all runs were indicated in Table 7.

TABLE 7
Parameters of Fraction II + III Extract Precipitation and Separation

| | | | Run 1 | | |
|---|---|---|---|---|---|
| Parameter | Unit | Run 1 | Control | Run 2 | Run 3 |
| Theoretical Wet Paste | [kg] | 4.29 | 4.29 | 4.78 | 5.14 |
| Yield | | | | | |
| Suspension pH (Initial) | [pH] | 5.0 | 5.1 | 4.99 | 4.96 |
| Temperature of Suspension | [° C.] | 3.9 | 4.0 | 4.1 | 3.9 |
| Acetic Acid in Extraction | [g/L] | 0.42 | 0.32 | 0.42 | 0.42 |
| Buffer | | | | | |
| Extraction Buffer Weight | [kg] | 64.5 | 83.8 | 71.7 | 77.3 |
| Buffer-to-Theoretical | N/A | 15.0 | 19.5 | 15.0 | 15.0 |
| Paste Ratio | | | | | |
| Extraction Time | [hh:mm] | 03:00 | 03:00 | 03:00 | 03:00 |
| Final pH | [pH] | 5.01 | 5.09 | 5.01 | 5.02 |
| Suspension Weight | [kg] | 72.3 | 92.4 | 81.8 | 87.9 |
| Separation Method | N/A | Eaton 400 | Eaton 400 | Eaton 400 | Eaton 400 |
| | | filter press | filter press | filter press | filter press |
| Filter Type | N/A | Cuno 50SA | Cuno 50SA | Cuno 50SA | Cuno 50SA |
| Effective Surface Area | [m²] | 0.88 | 0.99 | 0.99 | 0.99 |
| Filter Load | [L CP/m²] | 98.2 | 101.0 | 113.9 | 121.5 |
| Frame Thickness (single) | [mm] | 30 | 30 | 30 | 30 |
| Frame Volume (single) | [L] | 2.75 | 2.75 | 2.75 | 2.75 |
| Dead volume | [L] | 12.0 | 13.4 | 13.4 | 13.4 |
| Aerosil Target | [g/L CPP] | 2.3 | 2.3 | 2.3 | 2.3 |
| Aerosil Amount | [kg] | 0.2303 | 0.2302 | 0.2549 | 0.2750 |
| Aerosil Contact Time | [hh:mm] | 03:27 | 02:23 | 02:11 | 02:01 |
| Filteraid Type | N/A | Celpure C300 | Celpure C300 | Celpure C300 | Celpure C300 |
| Filteraid Target | [g/L] | 12 g/L CPC | 12 g/L CPP | 12 g/L CPC | 12 g/L CPC |
| Filteraid Amount | [kg] | 1.0368 | 1.2003 | 1.1495 | 1.2249 |
| Pre-Wash Vol (wash | [L] | 81.0 | 88.2 | 83.2 | 83.2 |
| solution) | | | | | |
| Post-Wash Vol (wash | [L] | 24.0 | 26.8 | 26.8 | 26.8 |
| solution) | | | | | |
| Temp (end of pre-wash) | [° C.] | 5.0 | 5.4 | 4.9 | 4.7 |
| Mean Filtration Temp | [° C.] | 4.6 | 4.5 | 4.3 | 4.5 |
| Max Pressure (filtration) | [bar] | 1.0 | 1.0 | 1.1 | 1.0 |
| Final Flux | [L/hr/m²] | 52.7 | 115.4 | 101.6 | 135.5 |
| Supernatant Density | [kg/L] | 0.999 | 0.999 | 0.999 | 0.999 |
| Total Supernatant Volume | [L] | 98.5 | 120.1 | 107.7 | 114.1 |
| Duration of Filtration | [hh:mm] | 01:20 | 00:45 | 00:32 | 00:30 |
| Duration of Filtration + | [hh:mm] | 02:07 | 01:03 | 01:04 | 00:51 |
| Post-Wash | | | | | |

The filtrate resulting from Fraction II+III precipitate was contacted with a detergent. forming a treated filtrate. After adjusting the pH of the treated filtrate to about 7.0, and adding ethanol to a final concentration of from about 20% to about 30%. a Precipitate G precipitate was formed. The technical parameters associated with Ppt G precipitation and separation from Fraction II+III precipitate in all runs were indicated in Table 8.

TABLE 8
Parameters of Ppt G Precipitation and Separation

| | | | Run 1 | | |
|---|---|---|---|---|---|
| Parameter | Unit | Run 1 | Control | Run 2 | Run 3 |
| pH Before Adjustment | [pH] | 6.55 | 6.36 | 6.38 | 6.40 |
| pH Before Alcohol | [pH] | 6.95 | 7.01 | 7.00 | 7.01 |
| Temperature Before | [° C.] | 1.5 | 2.0 | 1.1 | 1.8 |
| Alcohol | | | | | |
| Alcohol Add Time | [hh:mm] | 04:00 | 04:00 | 04:01 | 04:01 |
| Temperature After | [° C.] | −9 | −9 | −9 | −9 |
| Alcohol | | | | | |
| Final pH | [pH] | 7.20 | 7.23 | 7.11 | 7.07 |
| Total Aging Time | [hh:mm] | 10:11 | 10:11 | 12:05 | 12:14 |
| Suspension Weight | [kg] | 128.0 | 156.1 | 140.0 | 148.3 |
| Separation Method | N/A | CEPA Z61H | CEPA Z61H | CEPA Z61H | CEPA Z61H |
| Supernatant Density | [kg/L] | 0.971 | 0.971 | 0.971 | 0.971 |
| Total Supernatant | [L] | 126.5 | 157.4 | 141.3 | 149.9 |
| Volume | | | | | |
| Paste Weight | [kg] | 1.7436 | 1.7775 | 2.343 | 2.199 |
| Duration of | [hh:mm] | 02:44 | 03:26 | 01:46 | 01:53 |
| Centrifugation | | | | | |
| Average Flowrate | [L/h] | 46.3 | 45.8 | 80.0 | 79.6 |
| (approx) | | | | | |

### 3. Plasma Derived Products Isolated From Fraction IV and V of Fractioned Cohn Pool Were Not Affected by Cohn Pool Concertation

Proteins typically found in the Fraction IV and V downstream from Cohn pool concentration are found in these fractions in yields and purity comparable to those found in Fraction IV and V in a process starting with non-concentrated Cohn Pool. The conditions of exemplary separation and purification processes for those plasma derived products from Cohn Pool concentrate, including Albumin, Alpha-1 proteinase inhibitor, are set forth in Table 9-14.

The supernatant of Fraction II+III was further submitted to fractionation procedure. The supernatant of Fraction II+III was contacted with 25% ethanol to obtain Fraction IV-1 precipitate. The technical protocol and parameters for isolating protein in Fraction IV-1 were indicated in Table 9.

TABLE 9
Parameters of Fraction IV-1 Precipitation and Separation with 25% Ethanol

| | | | Run 1 | | |
|---|---|---|---|---|---|
| Parameter | Unit | Run 1 | Control | Run 2 | Run 3 |
| pH Before Aging | [pH] | 5.56 | 5.51 | 5.47 | 5.49 |
| Suspension Temp | [° C.] | −6.5 | −6.5 | −5.5 | −5.5 |
| Total Aging Time | [hh:mm] | 13:40 | 13:40 | 13:09 | 14:07 |
| Final pH | [pH] | 5.51 | 5.47 | 5.41 | 5.48 |
| Suspension Weight | [kg] | 127.3 | 148.1 | 140.3 | 147.1 |
| Separation Method | N/A | Eaton 400 | Eaton 400 | Eaton 400 | Eaton 400 |
| | | filter press | filter press | filter press | filter press |
| Filter Type | N/A | Becopad | Becopad | Becopad | Becopad |
| | | P270 | P270 | P270 | P270 |
| Effective Surface Area | [m²] | 0.55 | 0.66 | 0.66 | 0.77 |
| Filter Load | [L CP/m²] | 182.7 | 152.3 | 170.8 | 156.2 |
| Frame Thickness (single) | [mm] | 25 | 25 | 25 | 25 |
| Frame Volume (single) | [L] | 2.20 | 2.20 | 2.20 | 2.20 |
| Dead volume | [L] | 6.5 | 7.6 | 7.6 | 8.7 |
| Filteraid Type | N/A | Celpure C300 | Celpure C300 | Celpure C300 | Celpure C300 |
| Filteraid Target | [g/L] | 13 g/L CPC | 13 g/L CPP | 13 g/L CPC | 13 g/L CPC |
| Filteraid Amount | [kg] | 1.1233 | 1.3002 | 1.243 | 1.3263 |
| Pre-Rinse Vol (water) | [L] | 13.8 | 16.5 | 16.5 | 19.3 |
| Pre-Wash Vol (wash | [L] | 49.5 | 59.4 | 66.7 | 70.0 |
| solution) | | | | | |
| Post-Wash Vol (wash | [L] | 10.5 | 12.5 | 12.5 | 14.6 |
| solution) | | | | | |
| Temp (end of pre-wash) | [° C.] | −4.0 | −4.0 | −3.9 | −4.4 |
| Mean Filtration Temp | [° C.] | −5.0 | −5.2 | −4.7 | −4.7 |
| Max Sustained Pressure | [bar] | 1.5 | 0.9 | 1.0 | 0.9 |
| (filtration) | | | | | |
| Final Flux | [L/hr/m²] | 57.1 | 64.5 | 71.6 | 70.0 |
| Supernatant Density | [kg/L] | 0.978 | 0.977 | 0.979 | 0.979 |
| Total Supernatant Volume | [L] | 147.8 | 165.0 | 156.7 | 165.5 |
| Paste Weight | [kg] | 4.918 | 5.756 | 5.525 | 5.948 |
| Duration of Filtration | [hh:mm] | 03:55 | 03:18 | 02:55 | 02:33 |
| Duration of Filtration + | [hh:mm] | 04:46 | 03:56 | 03:22 | 03:07 |
| Post-Wash | | | | | |

The supernatant of Fraction IV-1 was subsequently contacted with 40% ethanol to obtain Fraction IV-4 precipitate. The technical parameters for isolating proteins in Fraction IV-4 were indicated in Table 10.

TABLE 10
Parameters of Fraction IV-4 Precipitation and Separation with 40% Ethanol

| | | | Run 1 | | |
|---|---|---|---|---|---|
| Parameter | Unit | Run 1 | Control | Run 2 | Run 3 |
| pH Before ISA | [pH] | 5.54 | 5.50 | 5.50 | 5.49 |
| pH Before Alcohol | [pH] | 5.76 | 5.79 | 5.70 | 5.88 |
| Temperature Before | [° C.] | −6.6 | −6.3 | −6.0 | −6.0 |
| Alcohol | | | | | |
| Alcohol Add Time | [hh:mm] | 05:00 | 05:05 | 05:00 | 05:00 |
| pH After Alcohol | [pH] | 5.82 → 5.96 | 5.82 → 5.96 | 5.80 → 5.93 | 5.83 → 5.94 |
| Temperature After | [° C.] | −5.8 | −6.2 | −6.1 | −6.0 |
| Alcohol | | | | | |
| Total Aging Time | [hh:mm] | 07:46 | 07:57 | 10:23 | 10:29 |
| Final pH | [pH] | 5.96 | 5.94 | 5.95 | 5.97 |
| Suspension Weight | [kg] | 179.5 | 201.1 | 192.1 | 202.41 |
| Separation Method | N/A | Eaton 400 | Eaton 400 | Eaton 400 | Eaton 400 |
| | | filter press | filter press | filter press | filter press |
| Filter Type | N/A | Cuno 70CP | Cuno 70CP | Cuno 70CP | Cuno 70CP |
| Effective Surface Area | [m²] | 0.66 | 0.77 | 0.77 | 0.88 |
| Filter Load | [L CP/m²] | 152.3 | 130.5 | 146.4 | 136.7 |
| Frame Thickness (single) | [mm] | 25 | 25 | 25 | 25 |
| Frame Volume (single) | [L] | 2.20 | 2.20 | 2.20 | 2.20 |
| Dead volume | [L] | 7.6 | 8.7 | 8.7 | 9.8 |
| Filteraid Type | N/A | Celpure C300 | Celpure C300 | Celpure C300 | Celpure C300 |
| Filteraid Target | [g/L] | 15.5 g/L CPC | 17.2 g/L CPP | 15.5 g/L CPC | 15.5 g/L CPC |
| Filteraid Amount | [kg] | 1.339 | 1.720 | 1.482 | 1.582 |
| Pre-Rinse Vol (water) | [L] | 14.9 | 17.3 | 17.3 | 19.8 |
| Pre-Wash Vol (wash | [L] | 47.8 | 59.7 | 57.9 | 59.4 |
| solution) | | | | | |
| Post-Wash Vol (wash | [L] | 15.2 | 17.4 | 17.4 | 19.6 |
| solution) | | | | | |
| Temp (end of pre-wash) | [° C.] | −4.4 | −4.4 | −4.6 | −5.1 |
| Mean Filtration Temp | [° C.] | −5.4 | −5.6 | −5.3 | −5.4 |
| Max Sustained Pressure | [bar] | 0.9 | 0.9 | 1.0 | 1.0 |
| (filtration) | | | | | |
| Final Flux | [L/hr/m²] | 87.0 | 93.8 | 76.9 | 104.3 |
| Supernatant Density | [kg/L] | 0.953 | 0.954 | Not measured | Not measured |
| Total Supernatant Volume | [L] | 196.0 | 223.9 | ~217.2 | ~230.0 |
| Paste Weight | [kg] | 4.059 | 4.376 | 4.803 | 4.861 |
| Duration of Filtration | [hh:mm] | 02:54 | 02:40 | 03:10 | 02:55 |
| Duration of Filtration + | [hh:mm] | 03:23 | 03:04 | 03:38 | 03:29 |
| Post-Wash | | | | | |

The supernatant of Fraction IV-4 was subsequently contacted with 40% ethanol to obtain Fraction V precipitate. Fraction V of the invention includes primarily albumin. The technical protocol and parameters for isolating proteins in Fraction V of fractionated Cohn Pool in all runs were indicated in Table 11. Comparable total protein content in fraction IV-4 between Run 1 and the non-concentrated control run have been observed, as shown in Table 12.

TABLE 11
Parameters of Fraction V Precipitation and Separation with 40% Ethanol

| | | | Run 1 | | |
|---|---|---|---|---|---|
| Parameter | Unit | Run 1 | Control | Run 2 | Run 3 |
| pH Before Buffer Add | [pH] | 5.90 | 5.87 | 5.94 | 5.97 |
| Temperature Before | [° C.] | −6.6 | −6.4 | −6.4 | −6.1 |
| Buffer Add | | | | | |
| Buffer Add Time | [hh:mm] | 06:00 | 06:00 | 06:00 | 06:00 |
| Total Aging Time | [hh:mm] | 10:27 | 10:27 | 12:34 | 13:15 |
| Final pH | [pH] | 4.80 | 4.78 | 4.78 | 4.79 |
| Temperature After Aging | [° C.] | −6.8 | −6.8 | −6.8 | −6.9 |
| Suspension Weight | [kg] | 191.0 | 218.4 | 211.0 | 223.8 |
| Separation Method | N/A | Eaton 400 | Eaton 400 | Eaton 400 | Eaton 400 |
| | | filter press | filter press | filter press | filter press |
| Filter Type | N/A | Becopad | Becopad | Ahlstrom | Ahlstrom |
| | | P270 | P270 | 950 | 950 |
| Effective Surface Area | [m²] | 0.66 | 0.77 | 0.99 | 1.10 |
| Filter Load | [L CP/m²] | 152.3 | 130.5 | 113.9 | 109.3 |
| Frame Thickness (single) | [mm] | 30 | 30 | 30 | 30 |
| Frame Volume (single) | [L] | 2.75 | 2.75 | 2.75 | 2.75 |
| Dead volume | [L] | 9.25 | 10.6 | 13.4 | 14.8 |
| Filteraid Type | N/A | N/A | N/A | N/A | N/A |
| Pre-Rinse Vol (water) | [L] | 14.9 | 17.3 | N/A | N/A |
| Pre-Wash Vol (wash | [L] | 50.6 | 59.1 | 66.9 | 83.3 |
| solution) | | | | | |
| Post-Wash Vol (wash | [L] | N/A | N/A | N/A | N/A |
| solution) | | | | | |
| Temp (end of pre-wash) | [° C.] | −4.9 | −5.1 | −6.0 | −6.0 |
| Mean Filtration Temp | [° C.] | −5.4 | −5.8 | −5.9 | −5.9 |
| Max Sustained Pressure | [bar] | 2.4 | 2.4 | 2.0 | 2.2 |
| (filtration) | | | | | |
| Final Flux | [L/hr/m²] | 47.4 | 53.1 | 109.6 | 39.4 |
| Supernatant Density | [kg/L] | 0.950 | 0.951 | 0.951 | 0.952 |
| Total Supernatant Volume | [L] | 143.4 | 187.2 | 219.3 | 232.0 |
| Remaining Suspension | [kg] | 54.8 | 40.3 | N/A | N/A |
| Weight ⁽¹⁾ | | | | | |
| Paste Weight | [kg] | 6.911 | 7.761 | 11.852 | 13.189 |
| Calculated Total Paste ⁽¹⁾ | [kg] | 9.693 | 9.518 | N/A | N/A |
| Duration of Filtration | [hh:mm] | 04:35 | 04:35 | 02:01 | 05:21 |

TABLE 12
Balance of Protein Isolated from Fraction IV-4

| | | Run 1 | |
|---|---|---|---|
| Parameters | Run 1 | Control | Delta |
| Cohn Plasma (Starting Volume) L | 100 | 100 | |
| Cohn Plasma for EtOH Frac L | 86.4 | 100 | |
| Total Protein (pool before conc.) g/l | | 50.5 | |
| Fr. IV4 Supern. Filtrate L | 196.1 | 223.9 | |
| Fr. IV4 Supern. Filtrate TP g/L | 12.9 | 11.5 | |
| Fr. IV4 Supern. Filtrate Total Protein g | 2529 | 2575 | |
| Fr. IV4 Supern. Filtrate g/L Alb./TP CP | 25.3 | 25.7 | −1.56% |
| (before conc.) | | | |

The quality of proteins isolated from Fraction V were evaluated through multiple biochemical and immunological assays as shown in Table 13. Quality indicating parameters of isolated proteins in Fraction V of Run 1 are aligned with the results of the respective non-concentrated control.

TABLE 13
Extended Characterization Testing at Fraction V

| Test Parameter | Unit | Run 1 | Run 1 Control |
|---|---|---|---|
| Albumin (Neph) | mg/mL | 52.250 | 52.700 |
| Haptoglobin | μg/mL | 263 | 330 |
| MSD - HPLC (Aggregates) | % area | 0.48 | 0.72 |
| MSD - HPLC (Dimers) | % area | 8.81 | 10.14 |
| MSD - HPLC (Fragments) | % area | 0.26 | 0.31 |
| MSD - HPLC (Monomers) | % area | 90.44 | 88.83 |
| PKA | IE/mL | <4 | <4 |
| Purity | % | 97.2 | 97.6 |
| Total Protein (Biuret) | mg/mL | 51.4 | 50.9 |
| Transferrin | μg/mL | 5.82 | 5.00 |
| al-Antitrypsin | mg/mL | <0.045 | <0.045 |

Fraction IV-1 intermediate isolated from concentrated Cohn Pool plasma was further processed until A1PI drug product. All relevant processing parameters are summarized in Table 14.

TABLE 14
Parameters of A1PI isolation and purification from Fraction IV-1

| | | | | Run 1/ | | |
|---|---|---|---|---|---|---|
| | | | Run 1 | Run 1 | | |
| Parameter | Unit | Run 1 | Control | Control % | Run 2 | Run 3 |
| CPP | L | 100.5 | 100.5 | 0.0 | 112.7 | 120.3 |
| FrIV-1 weighed in | g | 4197.2 | 5756.0 | −27.1 | 5525.0 | 5948.0 |
| fractionation | | | | | | |
| FrIV-1 suspended | g | 4165.7 | 4950.9 | −15.9 | 4966.0 | 4969.9 |
| CPP Eq | L | 99.7 | 86.4 | 15.4 | 101.3 | 100.5 |
| TP in suspension | g | 863.8 | 904.8 | −4.5 | 764.3 | 710.0 |
| AAT in suspension | g | 55.2 | 35.9 | 53.8 | 56.0 | 53.5 |
| AAT in suspension/CPP Eq | g AAT/L | 0.553 | 0.415 | 33.3 | 0.553 | 0.532 |
| | CPP | | | | | |
| AAT per kg of paste | g AAT/kg | 13.25 | 7.25 | 82.7 | 11.28 | 10.76 |
| | FrIV-1 | | | | | |
| Recovered AAT | g | 34.7 | 25.7 | 35.0 | 38.7 | 38.0 |
| after DEAE-1 | | | | | | |
| Recovered AAT/CPP Eq | g AAT/L | 0.349 | 0.298 | 17.0 | 0.383 | 0.379 |
| after DEAE-1 | CPP | | | | | |
| DEAE-1 Step Yield | % | 74.92 | 79.61 | −5.9 | 81.84 | 84.43 |
| Global Yield | % | 63.09 | 71.85 | −12.2 | 69.23 | 71.15 |
| Recovered AAT | g | 34.5 | 20.3 | 70.0 | 30.5 | 31.1 |
| after CM | | | | | | |
| Recovered AAT/CPP Eq | g AAT/L | 0.347 | 0.236 | 47.3 | 0.302 | 0.310 |
| after CM | CPP | | | | | |
| CM Step Yield | % | 95.52 | 80.40 | 18.8 | 84.67 | 87.41 |
| Global Yield | % | 63.33 | 57.37 | 10.4 | 55.08 | 58.84 |
| Recovered AAT | g | 26.7 | 14.9 | 79.2 | 23.2 | 23.9 |
| after DEAE-2 | | | | | | |
| Recovered AAT/CPP Eq | g AAT/L | 0.269 | 0.173 | 55.3 | 0.230 | 0.239 |
| after DEAE-2 | CPP | | | | | |
| DEAE-2 Step Yield | % | 82.45 | 82.33 | 0.1 | 81.25 | 82.34 |
| Global Yield | % | 50.08 | 43.92 | 14.0 | 42.93 | 46.41 |
| Recovered AAT | g | 23.2 | 10.8 | 114.8 | 18.6 | 19.3 |
| at Drug Substance | | | | | | |
| Recovered AAT/CPP Eq | g AAT/L | 0.236 | 0.127 | 86.4 | 0.186 | 0.195 |
| at Drug Substance | CPP | | | | | |
| Drug Substance | % | 96.51 | 88.61 | 8.9 | 88.41 | 92.09 |
| Step Yield | | | | | | |
| Global Yield until | % | 45.4 | 33.6 | 35.3 | 35.8 | 39.0 |
| Drug Substance | | | | | | |

Human Albumin and A1PI isolated from concentrated Cohn Pool plasma meet all predefined limits. All results of additional testing performed at drug product and intermediate level are within predefined ranges as shown in Table 15-17.

All results met the predefined acceptance criteria at intermediate and drug product level. Based on the illustrated results it can be concluded that Cohn Pool concentration does not negatively impact product quality of either A1PI or Albumin.

TABLE 15
Albumin Drug Product Test Results for Albumin Run 1 and Run 1 Control

| | | | Run 1 | | Run 1 Control | |
|---|---|---|---|---|---|---|
| | Acceptance | | Before | After | Before | After |
| Test | Criteria | Units | Pasteurization | Pasteurization | Pasteurization | Pasteurization |
| Physical | Clear, slightly | | Pass | Pass | Pass | Pass |
| Appearance | viscous liquid, | | | | | |
| | almost | | | | | |
| | colorless, | | | | | |
| | yellow, amber, | | | | | |
| | or green | | | | | |
| Total | 4.70 to 5.30 | g/dL | 5 | 5 | 4.9 | 4.9 |
| Protein | g/100 mL | | | | | |
| Protein | Albumin: 96% | % | 99% | 99% | 99% | 99% |
| Composition | minimum, | | | | | |
| (PCA) | Globulins: 4% | | 1% | 1% | 1% | 1% |
| | maximum | | | | | |
| N- | 0.064 to 0.096 | mmol/g | 0.083 | 0.083 | 0.081 | 0.08 |
| Acetyl | mmol/g | protein | | | | |
| Tryptophan | protein | | | | | |
| Octanoic | 0.064 to 0.096 | mmol/g | 0.067 | 0.067 | 0.078 | 0.077 |
| Acid | mmol/g | protein | | | | |
| | protein | | | | | |
| Sodium | 130 to 160 | mEq/L | 152 | 152 | 149 | 151 |
| | mEq/L | | | | | |
| Potassium | 2 mEq/L | mEq/L | <0.6 | <0.6 | <0.6 | <0.6 |
| | maximum | | | | | |
| Turbidity | Initial (heated | NU | initial 8 nu | initial 8 nu | initial 8 nu | initial 9nu |
| | 10 to 11 hrs. at | | heated 9 nu | heated 9 nu | heated 9 nu | heated 9 nu |
| | 60° C. ± 0.5° C.): | | | | | |
| | 30 N.U. | | | | | |
| | maximum | | | | | |
| | Heated (50 hrs. | | | | | |
| | at 57° C.): 30 | | | | | |
| | N.U. | | | | | |
| | maximum | | | | | |
| Heat | Shall remain | | SAT | SAT | SAT | SAT |
| Stability | visually | | | | | |
| | unchanged | | | | | |
| | after heating at | | | | | |
| | 57° C. for 50 | | | | | |
| | hours when | | | | | |
| | compared to | | | | | |
| | an unheated | | | | | |
| | sample from | | | | | |
| | the same lot | | | | | |
| Heme | A403 nm = | Absorbance | 0.04 | 0.03 | 0.04 | 0.03 |
| Content | 0.25 maximum | unit | | | | |
| PKA | | IU/mL | N/A | <4 | N/A | <4 |
| Ph | 6.85 to 7.05 | | 7 | 6.9 | 6.8 | 6.8 |
| | (6.4 to 7.4) | | | | | |

TABLE 16
Albumin Drug Product Test Results for Albumin Run 2 and Run 3

| | | | Run 2 | | Run 3 | |
|---|---|---|---|---|---|---|
| | | | Before | After | Before | After |
| Test | Acceptance Criteria | Units | Pasteurization | Pasteurization | Pasteurization | Pasteurization |
| Physical | Clear, slightly viscous liquid, | | Pass | Pass | Pass | Pass |
| Appearance | almost colorless, yellow, | | | | | |
| | amber, or green | | | | | |
| Total Protein | 4.70 to 5.30 g/100 mL | g/dL | 5 | 5 | 4.9 | 4.9 |
| Protein | Albumin: 96% minimum, | % | 99% | 99% | 99% | 99% |
| Composition | Globulins: 4% maximum | | 1% | 1% | 0% | 1% |
| (PCA) | | | | | | |
| N-Acetyl | 0.064 to 0.096 mmol/g protein | mmol/g | 0.085 | 0.085 | 0.081 | 0.08 |
| Tryptophan | | protein | | | | |
| Octanoic | 0.064 to 0.096 mmol/g protein | mmol/g | 0.073 | 0.073 | 0.077 | 0.076 |
| Acid | | protein | | | | |
| Sodium | 130 to 160 mEq/L | mEq/L | 154 | 149 | 147 | 145 |
| Potassium | 2 mEq/L maximum | mEq/L | <0.6 | <0.6 | <0.6 | <0.6 |
| Turbidity | Initial (heated 10 to 11 hrs. at | NU | initial 9 nu | initial 10 nu | initial 9 nu | initial 10 nu |
| | 60° C. ± 0.5° C.): 30 N.U. | | heated 10 nu | heated 11 nu | heated 10 nu | heated 11 nu |
| | maximum | | | | | |
| | Heated (50 hrs. at 57° C.): 30 | | | | | |
| | N.U. maximum | | | | | |
| Heat Stability | Shall remain visually | | SAT | SAT | SAT | SAT |
| | unchanged after heating at | | | | | |
| | 57° C. for 50 hours when | | | | | |
| | compared to an unheated | | | | | |
| | sample from the same lot | | | | | |
| Heme | A403 nm = 0.25 maximum | Absorbance | 0.05 | 0.05 | 0.07 | 0.06 |
| Content | | unit | | | | |
| PKA | | IU/mL | <4 | <4 | <4 | <4 |
| Ph | 6.85 to 7.05 (6.4 to 7.4) | | 6.9 | 6.9 | 7.0 | 7.0 |

TABLE 17
A1PI Drug Product Test Results

| | Acceptance | | | Run 1 | | |
|---|---|---|---|---|---|---|
| Test | Criteria | Units | Run 1 | Control | Run 2 | Run 3 |
| AAT Concentration | 16-24 mg/mL | mg/mL | 21 | 22 | 21 | 21 |
| AAT Activity | 0.7-1.1 mg | mg | 1.0 | 1.0 | 1.0 | 1.0 |
| | AAT/mg protein | AAT/mg | | | | |
| | | protein | | | | |
| Total Protein | 16-24 mg/mL | mg/mL | 20 | 23 | 21 | 21 |
| Gel Electrophoresis | Co migrates with | Pattern | conform to | conform to | conform to | conform to |
| | API Standard | | reference | reference | reference | reference |
| | | | profile | profile | profile | profile |
| Molecular Size | Monomers NLT | % | 100 | 100 | 100 | 100 |
| Distribution | 95% | | <1.0 | <0.3 | <0.3 | <0.3 |
| | Non-monomeric | | <0.5 | <0.5 | <0.5 | <0.5 |
| | forms NMT 5% | | | | | |
| | Fragments NMT | | | | | |
| | 1.6% | | | | | |
| S/D Detergents | Residual Tween | ppm | ≤20 | ≤20 | ≤20 | ≤20 |
| | NMT 20 ppm | | ≤5 | ≤5 | ≤5 | ≤5 |
| | Residual TNBP | | | | | |
| | NMT 5 ppm | | | | | |
| Residual PEG (UV | NMT 20 ppm | ppm | ≤20 | ≤20 | ≤20 | ≤20 |
| spectroscopy) | | | | | | |
| Appearance/ | Solution is clear | N/A | satisfactory | satisfactory | satisfactory | satisfactory |
| Visual Inspection | and colorless to | | | | | |
| | yellow-green | | | | | |
| | May contain a | | | | | |
| | few particles | | | | | |
| pH | 6.8-7.2 | — | 7.0 | 7.0 | 7.0 | 7.0 |
| Sodium | 130-170 | mEq/L | 168 | 161 | 160 | 154 |
| (Sodium) Chloride | 6-8 | mg/mL | 7 | 7 | 7 | 7 |
| Phosphate | 18-22 | mM | 21 | 21 | 21 | 21 |
| Isoelectric focus | Similar pattern to | Pattern | conform to | conform to | conform to | conform to |
| Gel | API Standard | | reference | reference | reference | reference |
| | | | profile | profile | profile | profile |
| Total Protein | 16-24 | mg/mL | 21 | 22 | 20 | 21 |
| (Bradford) | | | | | | |

### Example 3

A commercial scale run has been performed in present invention until Precipitate G (PptG), Fraction V (FrV) and Fraction IV-1 (Fr IV-1) intermediates. The desired Cohn Pool plasma volume and protein concentration were obtained without significant loss of various plasma derived proteins from different fractionations of Cohn Pool plasma.

PptG and Fr IV-1 were further purified at pilot scale to Immunoglobulin drug product and to A1PI drug product, respectively. FrV was purified at commercial scale to Albumin drug product.

Prior to Cohn fractionation process, about 4700 liters cryo-poor plasma were concentrated using an ultrafiltration membrane. For the plasma concentration step, pre-filtration (10 μm+0.5 μm) followed by batch TFF with UF cassettes with a nominal molecular weight cut off (NMWCO) of 100 kDa was employed. As a result, the starting protein concentrations of the Cohn Pool plasma in the test runs were about 15% higher than that in control run. For each protein of interest, technical specification of the separate and purified protein, for example, recovery rate, quality, are tabulated and analyzed.

### 1. Immunoglobulin Process Performance and Product Quality Were Not Affected by Cohn Pool Concentration

Immunoglobulin content was calculated following the concentration step. The resulting IgG content in the Cohn Pool plasma prepared for Ethanol treatment was concentrated without significant loss.

Proteins typically found in the Precipitate G and the Immunoglobulin drug product downstream from Cohn pool concentration are found comparable in yield and purity to those found in Precipitate G and Immunoglobulin drug product in a process starting with non-concentrated Cohn Pool.

All operational parameters for upstream and downstream processing such as cycle time and step yields were found comparable to historic commercial manufacturing data. IgG quality was evaluated at drug product level and all results met predefined limits as shown in Table 18.

TABLE 18
Immunoglobulin Drug Product Test Results

| Test | Acceptance Criteria | Units | Commercial Scale Run |
|---|---|---|---|
| Measles | ≥0.22 | times CBER Ref | 0.54 |
| Antibody | | Lot 176 | |
| Diphtheria | ≥1.2 U of US standard | U/mL | 9.4 |
| Antibody | Antitoxin/mL | | |
| Poliomyelitis | ≥0.2 | times the antibody | 0.8 |
| Antibody | | level of CBER | |
| | | Reference Polio | |
| | | Immune Globulin | |
| | | Lot 176 | |
| IgA | ≤0.14 | mg/mL | 0.05 |
| IgM | ≤10 | mg/dL | <4 |
| Anti-A | NMT 1/64 @25 g/L of IgG | 1/ | 32 |
| Anti-B | NMT 1/64 @25 g/L of IgG | 1/ | 16 |
| Anti-D | NMT NIBSC Ref. | — | Satisfactory |
| antibodies | 02/228 (or equivalent) | | |
| Anti- | NMT 50 corresponding | % | 42 |
| complement | to 1 CH50 U/mg protein | | |
| Activity | | | |
| Appearance | The liquid preparation is | — | Satisfactory |
| | clear or slighlty | | |
| | opalescent and colorless | | |
| | or pale yellow | | |
| Glycine | 0.20-0.30/0.21-0.26 | M | 0.23 |
| Purity | ≥98 | % | 100 |
| Osmolality | 240-300 | mOsmol/kg | 259 |
| pH | 4.6-5.1 Diluted @ | — | 4.9 |
| | 1% with 0.9% NaCl | | |
| Prekallikrein | ≤7 | — | <5 |
| Activator | | | |
| Activity | | | |
| Ig G Subclass | Similar to normal | complies | complies |
| Distribution | plasma | | |
| Complement- | ≥60 (cfr. EP) | % | 103 |
| mediated Lysis | | | |
| of Red Blood | | | |
| Cells | | | |

### 3. Process Parameters for Immunoglobulin Separation and Purification From Cohn Pool in Commercial Scale Run

Cohn Pool was concentrated by about 15% (w/v) by ultrafiltration, forming a first Cohn Pool concentrate. The technical protocol and parameters associated with the process of ultrafiltration are indicated in Table 19.

TABLE 19
Parameters for Cohn Pool Concentration Step

| Description | Unit | Commercial Scale Run |
|---|---|---|
| Cohn Pool mass before | [kg] | 4684 |
| concentration | | |
| Cohn Pool volume | [L] | 4565 |
| before concentration | | |
| Type of pre-filter | N/A | PP filters e.g., 10 μm + |
| | | 0.5 μm or equivalent |
| Pre-filter surface area | [m²] | 1.6 m² (filter area |
| | | selection dependent on |
| | | desired filtration time) |
| Pre-filtration time | [hh:mm] | 06:07 |
| Type of filter for | N/A | OMEGA T Series |
| concentration | | 100 kDa or equivalent |
| Filter surface area | [m²] | 30 |
| Number of filters used | N/A | 12 |
| Filtration/concentration | [hh:mm] | 1:01 |
| time | | |
| Maximum sustained TMP | [bar] | 1.5 |
| Average retentate flowrate | [L/min] | 8200 l/h |
| Average permeate flowrate | [g/min] | approx. 700 L/H |
| Initial concentration | [%] | 15% |
| factor (by mass) | | |
| Post-wash using 0.9% NaCl | [L] | 41 L (minimum |
| | | amount to clear line) |
| Cohn Pool mass after | [kg] | 3982.9 |
| concentration & post-wash | | |
| Cohn Pool volume after | [L] | 3882 |
| concentration & post-wash | | |
| Final concentration | [%] | 15% |
| factor (by mass) | | |
| Filter load | [L CPP/m²] | 152.2 |
| Turbidity - before conc./ | [NTU] | 332/258 |
| after conc. | | |

In the exemplary CPC process, target amounts of filter aid and filter area were calculated based on the volume of concentrated Cohn Pool instead on the volume of cryo-poor plasma (CPP).

The first Cohn Pool concentrate was further submitted to fractionation procedure. The first Cohn Pool concentrate was contacted with 8% ethanol at a pH of from about 7.0 to 7.5 to obtain a Fraction I precipitate and a Fraction I supernatant from the fractionated Cohn Pool.

The Fraction I supernatant was further contacted with about 25% ethanol at a pH of from about 6.7 to about 7.3 to form a Fraction II+III precipitate.

The Fraction II+III precipitate was further suspended in a suspension buffer, thereby forming an IgG suspension. After mixing finely divided silicon dioxide (SiO₂) with the IgG suspension for at least about 30 minutes, IgG suspension was filtered and resulted a filtrate and a filter cake.

The filtrate resulting from Fraction II+III precipitate was contacted with a detergent, forming a treated filtrate. After adjusting the pH of the treated filtrate to about 7.0, and adding ethanol to a final concentration of from about 20% to about 30%, a Precipitate G precipitate was formed.

### 3. Plasma Derived Products Isolated From Fraction IV and V of Fractioned Cohn Pool Were Not Affected by Cohn Pool Concertation

Proteins typically found in the Fraction IV and V downstream from Cohn pool concentration are found in these fractions in yields and purity comparable to those found in Fraction IV and V in a process starting with non-concentrated Cohn Pool.

The supernatant of Fraction II+III was further submitted to fractionation procedure. The supernatant of Fraction II+III was contacted with 25% ethanol to obtain Fraction IV-1 precipitate.

The supernatant of Fraction IV-1 was subsequently contacted with 40% ethanol to obtain Fraction IV-4 precipitate.

The supernatant of Fraction IV-4 was subsequently contacted with 40% ethanol to obtain Fraction V precipitate.

The quality of proteins isolated from Fraction V were evaluated through multiple biochemical and immunological assays as shown in Table 20. Quality indicating parameters of isolated proteins in Fraction V of the commercial scale run are comparable to historic commercial manufacturing data starting from non-concentrated Cohn Pool.

TABLE 20
Extended Characterization Testing at Fraction V

| Test Parameter | Unit | Commercial Scale Run |
|---|---|---|
| PKA | IU/mL | <4 |
| Purity | % | 97.2 |

Human Albumin and A1PI isolated from concentrated Cohn Pool plasma were tested at final drug product level. All results at drug product level are within predefined ranges as shown in Table 21-22.

All results met the predefined acceptance criteria at drug product level. Based on the illustrated results it can be concluded that Cohn Pool concentration does not negatively impact product quality of either A1PI or Albumin.

TABLE 21
20% Albumin Drug Product Test Results from Commercial Scale Run

| Test | Acceptance Criteria | Units | Commercial Scale Run |
|---|---|---|---|
| Visual Control | clear, slightly viscous | N/A | Confirm |
| | solution; almost | | |
| | colourless, yellow | | |
| | to brown or green | | |
| pH-Value | 6.7-7.3 | N/A | 7.0 |
| Potassium Content | ≤2 | μmol/mL | <0.9 |
| Aluminum Content | ≤200 | μg/L | ≤50 μg/L |
| Haem Content | <0.15 | Absorbance | 0.05 |
| | | unit | |
| Polymers and | Area of the peak due to | % | Polymers + |
| Aggregates (area %/ | polymers and aggregates is | | Aggregates 6% |
| protein %) | not greater than 10 percent | | Polymers + |
| | of the total area of the | | Aggregates |
| | chromatogram/corresponding | | (proteins) 3% |
| | to not more than 5% of | | |
| | polymers and aggregates | | |
| Prekallikrein | ≤35 IU/mL | IU/mL | <4 |
| Activator Activity | | | |
| Sodium Content | ≤160 | μmol/mL | 123 |
| Protein Composition | ≥96% human albumin | % human albumin | 98.4% |
| Protein Content | 20% Albumin: 190-210 | mg/mL | 196 |
| Heat Stability Test | complies (remain | Complies (remains | Complies |
| | unchanged) | unchanged) | |
| Bacterial Endotoxins | 20%: ≤1.7 | EU/mL | <0.5 |
| Test for Sterility | sterile | N/A | sterile |
| Particle | ≥10 μm: ≤6000 | particles/vial | ≥10 μm: 744 |
| Contamination | ≥25 μm: ≤600 | | ≥25 μm: 24 |
| Osmolality | 210-400 | mOsmol/kg | 226 |

TABLE 22
A1PI Drug Product Test Results

| | | | Commercial |
|---|---|---|---|
| Test | Acceptance Criteria | Units | Scale Run |
| AAT Concentration | 16-24 mg/mL | mg/mL | 21 |
| AAT Specific Activity | ≥0.35 mg AAT/mg | mg AAT/mg | 1.0 |
| | protein | protein | |
| Appearance/Visual | Solution is clear | N/A | satisfactory |
| Inspection | and colorless to | | |
| | yellow-green | | |
| | May contain a few | | |
| | protein particles | | |
| pH | 6.5-7.8 | — | 7.0 |

In additional lab scale filtration experiments it was demonstrated that permeate flux was comparable for filters up to 300 kDa molecular weight cut-off when concentrating Cohn Pool.

What is claimed is:

1. A method for preparing a Cohn Pool concentrate from blood plasma, the method comprising:
(a) providing a Cohn Pool from blood plasma; and
(b) concentrating the Cohn Pool to a total protein concentration of at least about 65 g/L, thereby forming a Cohn Pool concentrate.

2. The method of claim 1, wherein the Cohn Pool is concentrated to a protein concentration of at from about 50 to about 65 g/L

3. The method of any preceding claim, wherein the Cohn Pool is a member selected from cryo poor plasma, Factor poor plasma, and Inhibitor poor plasma.

4. The method of any preceding claim, wherein step (b) is performed using an ultrafiltration membrane with a nominal molecular weight cut off (NMWCO) of 300 kDa or less run either in re-circulation or in single-pass configuration, either with or without preceding pre-filtration.

5. The method of any preceding claim, wherein step (b) is performed using a hollow-fiber filter membrane with a nominal molecular weight cut off (NMWCO) of 300 kDa or less run either in re-circulation or in single-pass configuration, either with or without preceding pre-filtration.

6. The method of any preceding claim, wherein in the Cohn Pool concentrate has a total protein concentration of at least about 65 g/L.

7. The method of any preceding claim, wherein the Cohn Pool concentrate is further subjected to a purification process for preparing a composition selected from an immunoglobulin G (IgG) composition, an albumin composition, an A1-PI composition and a combination thereof.

8. The method of any preceding claim, wherein Cohn Pool is concentrated with essentially no loss of protein mass attributable to a member selected from Total Protein, IgG, albumin, AAT, and fibrinogen in the resulting Cohn Pool concentrate.

9. The method of any preceding claim, further comprising submitting the Cohn Pool concentrate to a plasma fractionation procedure.

10. The method of claim 9, wherein the fractionation procedure is Cohn Fractionation or one of its modifications.

11. The method of claim 10, wherein the fractionation procedure comprises:
(i) contacting the Cohn Pool concentrate with from about 6% to about 10% ethanol at a pH of from about 7.0 to 7.5 to obtain a Fraction I precipitate and a Fraction I supernatant; and
(ii) contacting the Fraction I supernatant or Cohn pool concentrated with from about 18% to about 27% alcohol at a pH of from about 6.7 to about 7.3 to form a member selected from a Fraction II+III precipitate or alternatively a Fraction I+II+III precipitate.

12. The method of claim 11, further comprising:
(iii) suspending a member selected from the Fraction II+III precipitate, the Fraction II+III (II+III or alternatively I+II+III) precipitate in a suspension buffer, thereby forming an IgG suspension;
(iv) mixing finely divided silicon dioxide (SiO₂) with the IgG suspension for at least about 30 minutes;
(v) filtering the IgG suspension, thereby forming a filtrate and a filter cake.

13. The method of claim 12, further comprising:
(vi) contacting the filtrate with a detergent, forming a treated filtrate;
(vii) adjusting the pH of the treated filtrate of step (vi) to about 7.0, and adding ethanol to a final concentration of from about 20% to about 30%, thereby forming a Precipitate G precipitate;
(viii) dissolving the Precipitate G precipitate in an aqueous solution comprising a member selected from a solvent a detergent and a combination thereof, forming a Precipitate G solution;
(ix) passing the solution through a cation exchange material, adsorbing proteins contained therein onto the cation exchange material, and subsequently eluting the adsorbed proteins in an eluate;
(x) passing the eluate through an anion exchange material generating a flow-through effluent;
(xi) passing the effluent through a nanofilter, generating a nanofiltrate;
(xii) concentrating the nanofiltrate by ultrafiltration, generating a first ultrafiltrate;
(xiii) diafiltering the first ultrafiltrate against a diafiltration buffer, generating a diafiltrate; and
(xi) concentrating the diafiltrate by ultrafiltration, generating a second ultrafiltrate having a protein concentration between about 8% (w/v) and about 22% (w/v), thereby forming an IgG enriched fraction.

14. The method of claim 13, further comprising, prior to (vi), washing the filter cake with at least 1 filter press dead volume of a wash buffer having a pH of from about 4.9 to about 5.3, thereby forming a wash solution; combining the filtrate with the wash solution, thereby forming a solution, and treating the solution with a detergent in step (vi).

15. A plasma protein preparation, wherein the protein is a member selected from IgG, A1PI, and albumin prepared by the method of claim 12.

16. A pharmaceutical formulation comprising albumin isolated from the Cohn Pool concentrate of claim 1, and a pharmaceutically acceptable vehicle.

17. A pharmaceutical formulation comprising AAT isolated from the Cohn Pool concentrate of claim 1, and a pharmaceutically acceptable vehicle.

18. A pharmaceutical formulation comprising IgG isolated from the Cohn Pool concentrate of claim 1, and a pharmaceutically acceptable vehicle.

19. A pharmaceutical formulation comprising fibrinogen isolated from the Cohn Pool concentrate of claim 1, and a pharmaceutically acceptable vehicle.

20. A pharmaceutical formulation comprising TP isolated from the Cohn Pool concentrate of claim 1, and a pharmaceutically acceptable vehicle.
